(19) United States
(12) Patent Application Publication
(10) Pub. No.: US 2025/0332442 A1
(43) Pub. Date: Oct. 30, 2025

(54) ORAL HYGIENE DEVICE AND SYSETM, AND USE METHOD

(71) Applicant: CHANGSHA EASYINSMILE INTELLIGENCE TECHNOLOGY CO., LTD., CHANGSHA (CN)

(72) Inventors: DAN GUO, CHANGSHA (CN); TIRAN ZHANG, CHANGSHA (CN)

(73) Assignee: CHANGSHA EASYINSMILE INTELLIGENCE TECHNOLOGY CO., LTD., CHANGSHA (CN)

(21) Appl. No.: 19/260,492

(22) Filed: Jul. 5, 2025

(30) Foreign Application Priority Data
May 27, 2023 (WO) PCT/CN2023/096697

(51) Int. Cl. A61N 5/06; A61B 18/00; A61K 31/5415; A61L 2/00; A61L 2/24; A61N 5/00

(52) U.S. Cl. CPC A61N 5/0624; A61K 31/5415; A61L 2/0052; A61L 2/24; A61N 5/062; A61B 2018/00172; A61B 2018/00702; A61B 2018/00827; A61L 2202/11; A61L 2202/14; A61N 2005/005; A61N 2005/0606; A61N 2005/0612; A61N 2005/0626; A61N 2005/0651; A61N 2005/0663

(57) ABSTRACT

Disclosed are an oral hygiene device and an oral hygiene device system, and a method of using the same. The oral hygiene device includes a housing, a battery, a control chip, a light source, and a light guide needle. Battery, control chip and light source are encapsulated in housing to form the oral hygiene device. The housing includes a connector, and light source is positioned in the center of connector. The light source is electrically connected to control chip and battery. The light guide needle is mounted on a head of the housing using a mortise-tenon structure. The light guide needle includes a mounting joint, and is in mortise-tenon connection and in interference fit with the connector through the mounting joint. The oral hygiene device has less damage to teeth or healthy tissue in the oral cavity, and has a good disinfection effect on bacteria in oral cavity.

### CROSS-REFERENCE TO RELATED APPLICATION

This patent application claims the priorities to Chinese Patent Application No. 202210584975.8 filed on May 27, 2022 and entitled “Method and device for dental caries plaque cleaning and periodontitis sterilization”, Chinese patent application No. 202221290282.X filed on May 27, 2022 and entitled “Device for dental caries plaque cleaning and periodontitis sterilization”, Chinese patent application No. 202210584980.9 filed on May 27, 2022 and entitled “Device and method for root canal sterilization and disinfection or whitening”, Chinese patent application No. 202221290080.5 filed on May 27, 2022 and entitled “Device for root canal sterilization and disinfection or whitening”, Chinese patent application No. 202211036511.X filed on Aug. 29, 2022 and entitled “Method and device for reducing peri-implantitis and periapical periodontitis reduction”, Chinese patent application No. 202211706409.6 filed on Dec. 29, 2022 and entitled “Optical power constant-current source control device and method for oral treatment”, and Chinese Patent application No. 202310599308.1 filed on May 25, 2023 and entitled “Oral hygiene device and system”.

### TECHNICAL FIELD

The present disclosure relates to an oral hygiene device and system, and a use method, and in particular to a device and system for oral photodynamic therapy, and a use method.

### BACKGROUND

Bacteria are easy to breed in the oral cavity, which may cause root canal inflammation, periodontitis, dental plaque, dental caries, pulpitis, and periodontitis. Photodynamic therapy (PDT) is a cold photochemical reaction involving light, photosensitizer and oxygen, which can produce singlet oxygen with strong oxidizing property to kill pathogenic microorganisms, and has the advantages of strong antibacterial effect, broad antibacterial spectrum, rapid action, good safety and no drug resistance. Some studies have shown that this method has a significant killing effect on oral bacteria. However, the use of photosensitizer may lead to discoloration of teeth, which is obvious in clinic. This is because the light sources used in PDT clinic at present are Er: YAG laser and Nd: YAG laser, which have high power and generate more heat, making more photosensitizers enter dentinal tubules and the color of teeth change obviously; moreover, the heat generation will cause irreversible damage to surrounding tissue, such as cementum damage, root resorption and alveolar bone necrosis.

### SUMMARY

An objective of the present disclosure is to provide an oral hygiene device, which has less damage to teeth or healthy tissues in an oral cavity, and has a good disinfection effect on bacteria in the oral cavity. Another objective of the present disclosure is to provide an oral hygiene system, which can further improve the disinfection effect of bacteria in the oral cavity. Still another objective of the present disclosure is to provide a use method of the oral hygiene device.

The technical objective above is achieved through the technical solution.

On one hand, the present disclosure provides a device for root canal sterilization and disinfection or whitening, including a housing, a battery, a control chip, an LED (light-emitting diode) working light source, and a root canal light guide needle.

The battery, the control chip and the LED working light source are encapsulated in the housing to form a device main body. The housing is further provided with a working switch, a treatment time selection button, and a light beam selection button. The working switch, the treatment time selection button and the light beam selection button are all electrically connected to the control chip.

The root canal light guide needle is mounted at the head of the housing using a mortise-tenon structure.

A head structure of the housing includes a connector, the LED working light source is arranged at the center of the connector, and is electrically connected to the control chip and the battery. The root canal light guide needle further includes a mounting joint, and is in mortise-tenon connection and in interference fit with the connector through the mounting joint.

According to the device for root canal sterilization and disinfection or whitening provided by the present disclosure, preferably, the connector is provided with four sections of clamping grooves, and the mounting joint is correspondingly provided with four sections of grooves, and the clamping grooves are clamped into the grooves, and the connector is connected to or disconnected from the mounting joint in an interference manner.

According to the device for root canal sterilization and disinfection or whitening provided by the present disclosure, preferably, the device further includes a charger, the battery is powered by the charger, and the LED working light source is powered by the battery. Rated optical power of the LED working light source is 25 mW. The LED working light source is a red and blue variable light source, where a wavelength of the red light ranges from 630 nm to 650 nm, and a wavelength of the blue light ranges from 430 nm to 450 nm.

According to the device for root canal sterilization and disinfection or whitening provided by the present disclosure, preferably, the root canal light guide needle is made of bisphenol A carbonate (PC for short), a front end of the root canal light guide needle is cone-shaped, and the optical power of a conical tip is 4.7 mW.

The present disclosure also provides a method for root canal sterilization and disinfection or whitening, including the following steps:
Step 1: when treating a root canal clinically, injecting normal saline into the root canal for rinsing, then taking out a root canal light guide needle to be mounted on a head of a housing;
Step 2: pressing and holding a working switch button on a device housing for 3 s to start the device; pressing a light beam selection button on the device housing until an upper indicator lamp shows red, indicating that a selected light beam wavelength is red light of 630 nm; pressing a treatment time selection button on the device housing until a lower indicator lamp shows white, indicating that the working time of a light source is 10 s; making the root canal light guide needle close to the root canal of a tooth, pressing the working switch button to start the light source for photodynamic sterilization, where the sterilization process is completed after 10 s; turning off the light source automatically, pressing the light beam selection button on the device housing until the upper indicator lamp shows blue light, indicating that the selected light beam wavelength is blue light of 450 nm, and irradiating the root canal of the tooth again for 10 s; and
Step 3: repeating Step two for three times, taking the device out of an oral cavity after the light source is automatically turned off, pressing and holding the working switch button on the device housing for 3 s to turn off the device, and finally, restoring the inherent shape and function of the tooth by filling a material or using other specific ways.

According to the method provided by the present disclosure, preferably, the working switch is configured to control the on and off of the device, and the on and off of the LED working light source. The treatment time selection button is configured to control the working time of the device, which may be 10 s, or 30 s. The working time of 10 s is mainly used for post-root canal therapy, and the working time of 30 s is mainly used for disinfection of periodontal bacteria. The light beam selection button is configured to control the wavelength of the LED working light source, which may be red light with the wavelength of 650 nm, or blue light with the wavelength of 450 nm. The red light with the wavelength of 650 nm is mainly used for sterilization and disinfection after root canal surgery; and the blue light with the wavelength 450 nm is mainly used for the whitening and cold light effect of the teeth.

According to the method for root canal sterilization and disinfection or whitening provided by the present disclosure, preferably, the red light is used for sterilization after root canal therapy: after root canal resection of the tooth in clinic, normal saline is firstly injected into a post-operative cavity for rinsing, and then a disposable root canal light guide needle is taken out and mounted at the head of the device. The red light of the device is selected as the auxiliary light, 0.1 mg/ml toluidine blue O photosensitizer is injected into the post-operative cavity with a syringe, the root canal light guide needle deeps into a coronal access cavity, and then the light source is started for photodynamic sterilization for 10 s. The device is taken out from the oral cavity after the light source is automatically turned off. Finally, a filling material is combined with the root canal dentin to complete the root canal therapy.

According to the method for root canal sterilization and disinfection or whitening provided by the present disclosure, preferably, the blue light is used for tooth whitening: when the tooth is whitened in clinic, the normal saline is first used to rinse the tooth, and a whitening agent and light-curing resin are applied to the tooth surface evenly, the blue light of the device is selected as auxiliary light, the device is close to the tooth surface, the light source is started to irradiate the tooth for 30 s, and then stands for 10 s, and such operations are repeated for three times. The device is taken out from the oral cavity after the light source is automatically turned off, thus completing the tooth whitening.

According to the method for root canal sterilization and disinfection or whitening provided by the present disclosure, preferably, a fuse mechanism is arranged in the control chip of the device, which is triggered after the light source continuously works for 2 min to make the LED working light source automatically stop, so as to prevent irreversible damage to gingiva caused by excessive red and blue light power and long-term irradiation.

According to the method for root canal sterilization and disinfection or whitening provided by the present disclosure, preferably, a relationship between a device supply voltage U and output power P of the root canal light guide needle is as follows:

$$P = \frac{1}{( 0.26 + 1.38 {e}^{- U} )} ;$$

e is a natural constant, a value of which is about 2.718281828 . . . .

A relationship between the output power P of the root canal light guide needle of the device and the efficiency E of killing Enterococcus faecalis is as follows:

$$E = \frac{48}{1 + [ {( P / 2.58 )}^{− 2.37} ]} + 58.9 .$$

On the other hand, a device for dental caries plaque cleaning and periodontitis sterilization is also provided, including a charger, a shell, a battery, a control chip, an LED working light source, and a periodontal light guide needle.

The battery, the control chip and the LED working light source are encapsulated in the housing to form a device main body. The shell is further provided with a working switch, a treatment time selection button, and a light beam selection button. The working switch, the treatment time selection button and the light beam selection button are all electrically connected to the control chip.

The periodontal light guide needle is mounted on a head of the shell by a mortise-tenon structure, a head structure of the shell includes a connector, the LED working light source is arranged at the center of the connector, and the LED working light source is electrically connected to the control chip and the battery.

The periodontal light guide needle further includes a mounting joint, and is in mortise-tenon connection and in interference fit with the connector through the mounting joint.

According to the device for dental caries plaque cleaning and periodontitis sterilization provided by the present disclosure, preferably, the connector is provided with four sections of clamping grooves, and the mounting joint is correspondingly provided with four sections of grooves, and the clamping grooves are clamped into the grooves, and the connector is connected to or disconnected from the mounting joint in an interference manner.

According to the device for dental caries plaque cleaning and periodontitis sterilization provided by the present disclosure, preferably, rated optical power of the LED working light source is 25 mW. The LED working light source is a red and blue variable light source, where a wavelength of the red light ranges from 630 nm to 650 nm, and a wavelength of the blue light ranges from 430 nm to 450 nm.

According to the device for dental caries plaque cleaning and periodontitis sterilization provided by the present disclosure, preferably, a front end of the periodontal light guide needle is cylindrical, the periodontal light guide needle has a length of 5 cm or 2 cm, and is configured to converge the light source. The optical power at a cylindrical tip is 12.8 mW or 15.4 mW.

According to the device for dental caries plaque cleaning and periodontitis sterilization provided by the present disclosure, preferably, the periodontal light guide needle is made of bisphenol A carbonate. Two models of periodontal light guide needles are provided, i.e., a long periodontal light guide needle and a short periodontal light guide needle, which are used for the treatment of internal and external periodontal diseases, respectively.

According to the device for dental caries plaque cleaning and periodontitis sterilization provided by the present disclosure, preferably, a relationship between a device supply voltage U and output power P of the periodontal light guide needle is as follows:

log(P)=0.53+0.78 log(U).

According to the device for dental caries plaque cleaning and periodontitis sterilization provided by the present disclosure, preferably, a relationship between the output power P of the periodontal light guide needle of the device and the efficiency E of killing Actinobacillus actinomycetemcomitans is as follows:

$$E = \frac{1 \text{?} 33}{1 + [ {( P / 10 )}^{− 2.5} ]} + 20.$$

$$\text{?} \text{indicates text missing or illegible when filed}$$

The present disclosure also provides a method for dental caries plaque cleaning and periodontitis sterilization, including the following steps:
Step 1: starting: first injecting normal saline into caries plaque for rinsing, and removing a device from a charger, and then taking out a disposable periodontal light guide needle to be mounted on a head of the device, where a long periodontal light guide needle and a short periodontal light guide needle are provided, the short periodontal light guide needle is used for external periodontal and the long periodontal light guide needle is used for internal periodontal; pressing and holding a working switch button on a shell for 3 s to start the device;
Step 2: for superficial caries:
S2.1: pressing a light beam selection button on the shell until an upper indicator lamp shows red, indicating that a selected light beam wavelength is red light, and pressing a treatment time selection button on the shell until a lower indicator lamp shows white, indicating that the working time of a light source is 10 s; making the periodontal light guide needle close to the caries plaque, pressing a working switch button to start the light source for photodynamic sterilization, where the sterilization process is completed after 10 s; turning off the light source automatically, pressing the light beam selection button on the shell until the upper indicator lamp shows blue, indicating that a selected light beam wavelength is blue light, and irradiating the caries plaque again for 10 s;
S2.2: repeating above operation for three times, taking the device out of the oral cavity after the light source is normally turned off, pressing and holding the working switch button on the shell for 3 s to turn off the device, and finally, restoring the inherent shape and function of the tooth by filling a material or using other specific ways, thus completing the treatment of superficial caries;
Step 3: for deep caries:
S3.1: pressing the light beam selection button on the shell until the upper indicator lamp shows red, indicating that the selected light beam wavelength is red light; pressing the treatment time selection button on the shell until the lower indicator lamp shows yellow, indicating that the working time of the light source is 30 s; and then injecting 0.1 mg/ml toluidine blue O photosensitizer into a deep caries cavity with a syringe;
S3.2: deeply extending the periodontal light guide needle into the deep caries cavity, pressing the working switch button to start the light source for photodynamic sterilization, where the sterilization process is completed after 30 s; turning off the light source normally, taking the device out of the oral cavity, pressing and holding the working switch button on the shell for 3 s to turn off the device, and finally, restoring the inherent shape and function of the tooth by filling a material or using other specific ways, thus completing the treatment of deep caries;
Step 4: for periodontitis:
where an operation for treating periodontitis is consistent with the operation for deep caries, the toluidine blue O photosensitizer is applied to a lesion site, and then the red light is used for photodynamic therapy; and
Step 5: if the light source continues to work for 2 min due to an unexpected situation in Step 2 or Step 3, triggering a fuse mechanism of a control chip, which makes the LED working light source stop automatically, thus preventing irreversible damage to gingiva caused by excessive red and blue light power and long-term irradiation.

Yet on the other hand, the present disclosure also provides a device for peri-implantitis and periapical periodontitis elimination, including a shell, a battery pack, a control chip, an LED working light source, and a pure flat light guide working tip.

The battery pack and the control chip are encapsulated in the housing to form a device main body, the battery pack is encapsulated at the bottom in the housing, and the control chip is encapsulated at a handle in the housing.

A working panel of the handle of the shell is provided with a working switch, a treatment time selection button, and a light beam selection button. The working switch, the treatment time selection button and the light beam selection button are all electrically connected to the control chip.

A head structure of the shell includes a connector, the LED working light source is arranged on the connector, and the LED working light source is electrically connected to the control chip and the battery pack, respectively.

The pure flat light guide working tip is mounted on the connector using a mortise-tenon structure, and a front end of the pure flat light guide working tip is in the shape of a frustum of a cone. The pure flat light guide working tip has a length of 2 cm, and is used for irradiating the light source.

According to the device for peri-implantitis and periapical periodontitis elimination, preferably, the device further includes a charger, the charger is arranged at the bottom of the shell and is embedded with the shell. The charger provides an electrode to be electrically connected to the battery pack.

According to the device for peri-implantitis and periapical periodontitis elimination, preferably, the bottom of the pure flat light guide working tip is further provided with a mounting joint, and is in mortise-tenon connection and in interference fit with the connector through the mounting joint.

According to the device for peri-implantitis and periapical periodontitis elimination, preferably, the connector is provided with four sections of clamping grooves, and the mounting joint is correspondingly provided with four sections of grooves, and the clamping grooves are clamped into the grooves, and the connector is connected to or disconnected from the mounting joint in an interference manner.

According to the device for peri-implantitis and periapical periodontitis elimination, preferably, the pure flat light guide working tip is made of bisphenol A carbonate, and is configured to irradiate lesions of peri-implantitis and periapical periodontitis.

According to the device for peri-implantitis and periapical periodontitis elimination, preferably, rated optical power of the LED working light source is 25 mW. The LED working light source is a red-blue variable light source, where a wavelength of the red light ranges from 630 nm to 650 nm, and a wavelength of the blue light ranges from 430 nm to 450 nm.

According to the device for peri-implantitis and periapical periodontitis elimination, preferably, a relationship between a battery pack supply voltage U and output power P of the pure flat light guide working tip is as follows:

$$log ( p ) = 0.45 + 0.83 log ( U ) .$$

According to the device for peri-implantitis and periapical periodontitis elimination, preferably, a relationship between the output power P of the pure flat light guide working tip and the efficiency of killing Porphyromonas gingivalis is as follows:

$$E = \frac{133}{1 + {( \frac{1}{p} )}^{3 / 2}} + 20.$$

The present disclosure also provides a method for peri-implantitis and periapical periodontitis elimination, including the following steps:
Step 1: symptom identifying:
S1.1: when a patient suffers from periapical periodontitis, treating according to Step 2;
S1.2: when a patient suffers from periodontitis, treating according to Step 3; and
S1.3: when a patient suffers from both periapical periodontitis and periodontitis, treating in the order of Step 2 and Step 3;
Step 2: for periapical periodontitis:
S2.1: pre-operation: first, fully expose a lesion after the incision of gingiva at a corresponding site, and carrying out drainage operation to remove pus, blood, etc. on the surface of the lesion, and then rinsing with normal saline;
S2.2: starting: removing a device from a charger, then taking out a disposable pure flat light guide working tip to be mounted on a head of the device, where the pure flat light guide working tip is a replaceable device shaped in the shape of a frustum of a cone and can act on the surface of the lesion after incision; and pressing and holding a working switch button on the shell for 3 s to start the device;
S2.3: pressing a light beam selection button on the shell until an upper indicator lamp shows red, indicating that a selected light beam wavelength is red light; pressing a treatment time selection button on the shell until a lower indicator lamp shows yellow, indicating that the working time of the light source is 30 s; and then injecting 0.1 mg/ml toluidine blue O photosensitizer into the incision using a needle-free syringe; and
S2.4: placing the pure flat light guide working tip right near the lesion, pressing the working switch button to start the light source for photodynamic sterilization, where the sterilization process is completed after 30 s; turning off the light source normally, taking the device out of an oral cavity, pressing the working switch button on the shell for 3 s to stop power supply, and then closing the lesion incision on the gingiva, and disinfecting, assisted by corresponding analgesic drugs;
Step 3: for periodontitis:
S3.1: sucking pus and rinsing a lesion region with normal saline;
S3.2: starting: removing the device from the charger, then taking out the disposable pure flat light guide working tip to be mounted on the head of the device, where the pure flat light guide working tip is a replaceable device shaped in the shape of a frustum of a cone and can act on the surface of the lesion after incision; and pressing and holding the working switch button on the shell for 3 s to start the device;
S3.3: pressing the light beam selection button on the shell until the upper indicator lamp shows red, indicating that a selected light beam wavelength is red light; pressing the treatment time selection button on the shell until the lower indicator lamp shows yellow, indicating that the working time of the light source is 30 s; and then injecting 0.1 mg/ml toluidine blue O photosensitizer into a periodontal pocket using a needle-free syringe;
S3.4: slightly spreading the periodontal pocket using tweezers, making the pure flat light guide working tip close to the photosensitizer at the periodontal pocket as much as possible, and pressing the working switch button to start the light source for photodynamic sterilization, where the sterilization process is completed after 30 s; turning off the light source normally, taking the device out of the oral cavity, and pressing and holding the working switch button on the shell for 3 s to stop the power supply; and
S3.5: if the light source continues to work for 2 min due to an unexpected situation, triggering a fuse mechanism of a control chip, which makes the LED working light source stop automatically, thus preventing irreversible damage to gingiva caused by excessive red and blue light power and long-term irradiation.

Further on the other hand, an optical power constant-current source control device for oral treatment is also provided, including a charging base, a plastic shell, a constant-current source circuit, a control chip, an LED working light source, a light guide head, a control circuit, and a power attenuation sheet.

The constant-current source circuit, the control chip and the control circuit are encapsulated in the plastic shell.

A hand-held section of the plastic shell is also provided with a control region including a working switch, a treatment time selection button and a light source wavelength selection button. The control region is electrically connected to the control chip.

The top of the plastic shell is provided with a concave connector, the LED light source is arranged at the center of the connector, and is electrically connected to the working switch, the light source wavelength selection button, and the control chip.

The power attenuation sheet is fixedly arranged at a front end of the LED light source. The power attenuation sheet is of a double-layer structure, including an inner fixed sheet and an outer rotary sheet which are coaxially arranged, each of the inner fixed sheet and the outer rotary sheet is composed of six 30° fan-shaped blades rotating in an array at intervals of 30° from each other.

The inner fixed sheet is fixed to the front end of the light source, the outer rotary sheet rotates around the center of the inner fixed sheet by 0-30°, and forms three different types of shading regions for the light source with the inner fixed sheet, which are complete shading, partial shading and minimum shading, and thus three different output power states are formed to achieve regulation of the optical power.

Three models of light guide heads with different specifications and applications are provided, i.e., a short periodontal light guide needle, a long root canal light guide needle, and a blunt flat light guide head.

Optical power of the periodontal light guide needle is from 60 mW to 85 mW, and the power attenuation sheet is in partial shading; optical power of the root canal light guide needle is about 40 mW to 50 mW, and the power attenuation sheet is in complete shading; and optical power of the blunt flat light guide needle is from 160 mW to 220 mW, and the power attenuation sheet is in minimum shielding.

The light guide head is fixedly mounted on the connector.

According to the optical power constant-current source control device provided by the present disclosure, preferably, an edge of the concave connector is a circular arc, the circular arc is designed with at least two clamping grooves, and the light guide head is in mortise-tenon connection and in interference fit with the clamping grooves through a corresponding convex mounting joint.

According to the optical power constant-current source control device provided by the present disclosure, preferably, the rated optical power of the constant-current source circuit is 500 mW to 700 mW. The LED working light source is a red-blue variable light source, where a wavelength of the red light ranges from 630 nm to 650 nm, and a wavelength of the blue light ranges from 430 nm to 450 nm.

According to the optical power constant-current source control device according to the present disclosure, preferably, the control chip further includes a current control chip for detecting the current in the circuit, and when the current is excessive, information about excessive current is fed back to the control chip to turn off the switch, so as to switch off the power supply circuit.

According to the optical power constant-current source control device provided by the present disclosure, preferably, the control chip further includes a temperature control chip and an indicator lamp, and the temperature control chip is linked with the indicator lamp through a switch. When the temperature control chip monitors that the temperature is more than 80° C., information about the temperature is fed back to the control chip to turn off the switch, so as to switch off the power supply circuit. After the temperature control chip monitors that the temperature drops to 40° C., information about the temperature is fed back to the control chip to turn on the switch to restore the power supply of the power supply circuit, which makes the indicator lamp flash to prompt continued use.

According to the optical power constant-current source control device provided by the present disclosure, preferably, the light guide head is made of bisphenol A carbonate.

The present disclosure also provides an optical power constant-current source control method for oral treatment, including the following steps:
Step 1: in an aerobic environment, attaching a toluidine blue O photosensitizer to bacteria, where the photosensitizer is in a singlet state at this time;
Step 2: after being irradiated by an external light source, exciting the photosensitizer to transition to an excited singlet state, and then quickly transitioning to an excited triplet state;
Step 3: enabling the triplet state of the photosensitizer to directly collide with tissue oxygen to release energy and return to the singlet state, where the released energy is absorbed by the tissue oxygen to generate singlet reactive oxygen; and
Step 4: producing, by reactive oxygen species (ROS), rapid lipid oxidation on bacteria, which affects the metabolism of the bacteria and leads to rapid death of the bacteria, thus achieving sterilization and anti-inflammatory effects.

The optical power constant-current source control method includes the following methods:
Method 1. Sterilization operation for periodontitis, dental plaque and caries is as follows:
S1.1: rinsing an operation space, and then drying the water in the oral cavity as much as possible with biological cotton;
S1.2: mounting a short periodontal light guide needle as a light guide head, pressing and holding a working switch on a plastic shell for 1 s until an indicator lamp flashes alternately in red and blue, then pressing a light source wavelength selection button to select a light source, where the red light is used for superficial anti-inflammation and dental plaque, and it is indicated that the system is ready when observing that the indicator lamp turns red and always lights up;
S1.3: applying the photosensitizer toluidine blue O (TBO for short) on the periodontal surface, extending a periodontal light guide head into the oral cavity, then pressing the working switch again to make the system start to work, where the single irradiation lasts for 30 seconds, and the periodontal light guide head rotates around the tooth lesion for irradiation during the process; and after cleaning, cleaning the photosensitizer in the oral cavity with normal saline until there is no color attached;
S1.4: after the sterilization process is completed after 30 s, turning off the light source normally, taking the device out of the oral cavity, and pressing and holding the working switch button on the shell for 1 s to stop the power supply.
Method 2. Sterilization operation in dental pulp and root canal is as follows:
S2.1: rinsing an operation space, and then drying the water in the oral cavity as much as possible with biological cotton;
S2.2: replacing the light guide head with a long periodontal light guide needle, pressing and holding the working switch on the plastic shell for 1 s until the indicator lamp flashes alternately in red and blue, then pressing the light source wavelength selection button to select the light source, where for normal root canal filling and cleaning, the blue light should be used for directly irradiating the lesion without using toluidine blue O for cooperation if the patient is in special circumstances such as no lesion, and it is indicated that the system is ready when observing that the indicator lamp turns blue and always lights up;
S2.3: injecting the photosensitizer toluidine blue O (TBO for short) into the root canal with a needle tubing, for deep root canal, producing liquid bubbles using a root canal scrubber after the injection, then extending the root canal light guide head into the oral cavity to align with the root canal of the tooth, pressing the working switch again to make the system start to work, where the duration of single irradiation is 60 s, and an inflammatory site should be irradiated emphatically during the process; and after cleaning, cleaning the root canal with the normal saline until there is no color attached; and
S2.4: after the sterilization process is completed after 60 s, turning off the light source normally, taking the device out of the oral cavity, and pressing and holding the working switch button on the shell for 1 s to stop the power supply.
Method 3. Sterilization operation for peri-implantitis is as follows:
S3.1: sucking pus, and rinsing a lesion region with normal saline;
S3.2: replacing the light guide head with a blunt flat light guide head, pressing and holding the working switch on the plastic shell for 1 s until the indicator lamp flashes alternately in red and blue, and then pressing the light source wavelength selection button to select a light source, where the red light should be used for irradiating the lesion for the peri-implantitis, applying the photosensitizer toluidine blue O to the lesion site of the peri-implantitis, injecting the photosensitizer toluidine blue O into a gingival surface with a syringe, extending the light guide head into the oral cavity to align with the lesion of the peri-implantitis, and then pressing the working switch again, where the duration of single irradiation is 90 s, and an inflammatory site should be irradiated emphatically during the process; and after cleaning, cleaning the photosensitizer in the oral cavity with normal saline until there is no color attached; and
S3.3: after the sterilization process is completed after 90 s, turning off the light source normally, taking the device out of the oral cavity, and pressing and holding the working switch button on the shell for 1 s to stop the power supply.
Method 4. Sterilization operation for dental periapical periodontitis is as follows:
S4.1: localizing a lesion, sucking pus and rinsing a lesion region with normal saline;
S4.2: replacing the light guide head as a blunt flat light guide head, pressing and holding the working switch on the housing for 1 s until the indicator lamp flashes alternately in red and blue, and then pressing the light source wavelength selection button to select a light source, where the red light is used for irradiating a lesion for dental periapical periodontitis; applying the photosensitizer toluidine blue O to a lesion site of the gingival surface, extending the light guide head into the oral cavity to align with the lesion of the periodontitis, and then pressing the working switch again to make the system start to work, where the duration of single irradiation is 90 s; irradiating an incision site emphatically during the process; and after cleaning, cleaning the photosensitizer in the oral cavity with normal saline until there is no color attached; and
S4.3: after the sterilization process is completed after 90 s, turning off the light source normally, taking the device out of the oral cavity, and pressing and holding the working switch button on the shell for 1 s to stop the power supply.

According to the optical power constant-current source control method provided by the present disclosure, preferably, a relationship between output power P of the light guide head and the efficiency E of killing Porphyromonas gingivalis and other pathogenic bacteria is as follows:

$$E = {( \frac{1}{p} )}^{- 3 / 2} + 15.$$

A relationship between the output power P of the light guide head and the efficiency E of killing Enterococcus faecalis is as follows:

$$E = \frac{35}{{( \frac{1}{p} )}^{\frac{7}{2}} - 1} .$$

In another aspect, an oral hygiene device is also provided by the present disclosure, including a main board, a metal substrate, a light source, a heat dissipation plate, a heat dissipation cover, a first cooling fin, and a second cooling fin.

The main board is provided with a control chip, and a control circuit.

The metal substrate is provided with a light source mounting surface, the light source is arranged on the light source mounting surface, and the metal substrate is electrically connected to the main board.

The heat dissipation plate is in contact with a surface, opposite to the light source mounting surface, of the metal substrate.

The heat dissipation cover is in contact with the light source mounting surface.

The first cooling fin and the second cooling fin are arranged on two opposite sides of the main board, respectively.

According to the device provided by the present disclosure, preferably, a light source sleeve and a working sleeve are further included.

The metal substrate is provided with an elastic sheet, and the elastic sheet is provided to control the main board to be connected to and disconnected from the light source.

The heat dissipation cover is provided with an elastic sheet opening, at least a part of a projection of the elastic sheet opening on a plane where the light source mounting surface coincides with a projection of the elastic sheet on the plane where the light source mounting surface.

The light source sleeve is sleeved outside the metal substrate, the heat dissipation plate and the heat dissipation cover. The light source sleeve is provided with a light source hole, and an elastic sheet button. The light source hole is provided for the transmission of light generated by the light source, and the elastic sheet button is arranged to touch the elastic sheet under the action of an external force.

The working sleeve is provided with a working tip connecting hole, and the working tip connecting hole is used for being connected to the working tip. The working tip connecting hole is provided for the transmission of the light generated by the light source. When the working sleeve is sleeved on a periphery of the light source sleeve, the working sleeve touches the elastic sheet button.

According to the device provided by the present disclosure, preferably, a projection of the light source on the plane where the light source mounting surface is located falls within the range of a projection of the light source hole on the plane where the light source mounting surface is located.

When the working sleeve is sleeved on the periphery of the light source sleeve, the projection of the light source on the plane where the light source mounting surface is located falls within the range of a projection of the working tip connecting hole on the plane where the light source mounting surface is located.

According to the device provided by the present disclosure, preferably, the heat dissipation plate is provided with a heat dissipation plate body and a heat dissipation plate connecting part. The heat dissipation plate body is provided to be attached to a surface, opposite to the light source mounting face, of the metal substrate. In a direction perpendicular to the light source mounting surface, a thickness of the heat dissipation plate connecting part is greater than that of the heat dissipation plate body, thus making the heat dissipation plate connecting part form a bulge on one side where the metal substrate is located. The metal substrate is located on the bulge formed by the heat dissipation plate connecting part.

The heat dissipation cover is provided with a heat dissipation cover body and a heat dissipation cover connecting part. The heat dissipation cover body is provided to cover the light source mounting surface, and the heat dissipation cover body does not shade the light source. The heat dissipation cover connecting part is connected to the heat dissipation plate connecting part.

According to the device provided by the present disclosure, preferably, the light source mounting surface falls into a projection of the heat dissipation plate body on the plane where the light source mounting surface is located, or the light source mounting surface coincides with the projection of the heat dissipation plate body on the plane where the light source mounting surface is located.

The light source mounting surface falls into a projection of the heat dissipation cover body on the plane where the light source mounting surface is located, or the light source mounting surface coincides with the projection of the heat dissipation cover body on the plane where the light source mounting surface is located.

According to the device provided by the present disclosure, preferably, the first cooling fin is provided with a first cooling fin connecting part, and a first cooling fin main board accommodating part. The first cooling fin connecting part is provided to be connected to the heating dissipation plate connecting part, the surface area of the first cooling fin is greater than that of the heat dissipation plate and is greater than that of the heat dissipation cover.

The second cooling fin is provided with a second cooling fin connecting part and a second cooling fin main board accommodating part. The second cooling fin connecting part is provided to be connected to the heating dissipation plate connecting part, the surface area of the second cooling fin is greater than that of the heat dissipation plate and is greater than that of the heat dissipation cover.

The first cooling fin main board accommodating part forms a main board accommodating cavity with the second cooling fin main board accommodating part, and a main board is located in the main board accommodating cavity.

According to the device provided by the present disclosure, preferably, the main board accommodating cavity is provided with a first cavity part, a second cavity part, and a third cavity part in communication in turn. The first cavity part is close to the first cooling fin connecting part and the second cooling fin connecting part.

In the first cavity part, a distance between the first cooling fin and the second cooling fin is denoted by F. In the second cavity part, a distance between the first cooling fin and the second cooling fin is denoted by S. In the third cavity part, a distance between the first cooling fin and the second cooling fin is denoted by T, T is less than S and is greater than or equal to F.

According to the device provided by the present disclosure, preferably, the light source is set to emit blue light or red light. The heat dissipation plate, the heat dissipation cover, the first cooling fin and the second cooling fin are made of metal materials.

According to the device provided by the present disclosure, preferably, the device further includes a battery, a charging interface, a shell, and a control button.

The charging interface is electrically connected to the battery, and is provided to supply electric energy to the battery.

The battery is electrically connected to the main board, and is provided to store electric energy and to supply electric energy to the main board.

The battery, the main board, the first cooling fin, the second cooling fin, and at least part of the charging interface are located in a cavity formed by the shell.

The charging interface is provided with a power supply connecting terminal, the shell is provided with a shell opening, and the power supply connecting terminal is exposed to the outside through the shell opening.

The control button is arranged on the shell, is connected to the control chip, and is provided to be able to control the lighting and extinguishing of the light source, the lighting time of the light source and a wavelength of the light emitted by the light source.

An oral hygiene system is also provided by the present disclosure, including the device above and a compound shown in Formula (I):

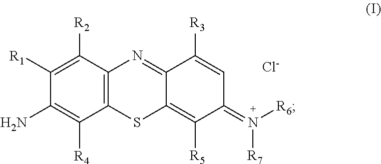

where R₁, R₂, R₃, R₄, R₅, R₆ and R₇ are independently selected from H or C1-C6 alkyl, respectively.

An oral hygiene device is also provided by the present disclosure, including a housing, a battery, a control chip, a light source, and a light guide needle.

The battery, the control chip and the light source are encapsulated in the housing to form a device main body.

The head of the housing is provided with a connector, the light source is arranged at the center of the connector, and the light source is electrically connected to the control chip and the battery.

The light guide needle is mounted at the head of the housing using a mortise-tenon structure, and the light guide needle is provided with a mounting joint, and is in mortise-tenon connection and in interference fit with the connector through the mounting joint.

According to the device provided by the present disclosure, preferably, the light source is a red-blue variable light source, where a wavelength of red light ranges from 630 nm to 650 nm, and a wavelength of blue light ranges from 430 nm to 450 nm. The light source is an LED working light source.

The light guide needle is made of bisphenol A polycarbonate, and is selected from one of the following light guide needles:
(1) a root canal light guide needle, wherein a front end of the root canal light guide needle is cone-shaped, and optical power at a conical tip is 4.7 mW;
(2) a periodontal light guide needle, wherein a front end of the periodontal light guide needle is cylindrical and is used for converging the light source, and optical power at a cylindrical tip is 12.8 mW or 15.4 mW; and
(3) a pure flat light guide needle, wherein a front end of the pure flat light guide needle is in the shape of a frustum of a cone.

According to the device provided by the present disclosure, preferably, the device further includes a power attenuation sheet, which is arranged at a front end of the light source.

The power attenuation sheet is of a double-layer structure, comprises an inner fixed sheet and an outer rotary sheet in coaxial arrangement. Each of the inner fixed sheet and the outer rotary sheet is composed of six 30° fan-shaped blades rotating in an array at intervals of 30° from each other. The inner fixed sheet is fixed to the front end of the light source, the outer rotary sheet rotates around the center of the inner fixed sheet by 0-30°, and forms three different types of shading regions for the light source with the inner fixed sheet, which are complete shading, partial shading and minimum shading, and thus three different output power states are formed to achieve regulation of the optical power.

According to the device provided by the present disclosure, preferably, three models of light guide needles with different specifications and applications are provided, i.e., a short periodontal light guide needle, a long root canal light guide needle, and a pure flat light guide needle.

Optical power of the periodontal light guide needle is from 60 mW to 85 mW, and the power attenuation sheet is in partial shading. Optical power of the root canal light guide needle is about 40 mW to 50 mW, and the power attenuation sheet is in complete shading. Optical power of the pure flat light guide needle is from 160 mW to 220 mW, and the power attenuation sheet is in minimum shielding.

According to the device provided by the present disclosure, preferably, the device further includes a constant current circuit, which is encapsulated in the housing. The rated output optical power of the constant current circuit is 500 mW to 700 mW.

According to the device provided by the present disclosure, preferably, the device further includes a current control chip, and/or a temperature control chip.

The current control chip is used for detecting a current in a power supply circuit, and when the current is excessive, information about excessive current is fed back to the control chip to turn off the switch, so as to switch off the power supply circuit.

The temperature control chip is linked with an indicator lamp through a switch; when the temperature control chip monitors that a temperature of the device is greater than 80° C., information about the temperature is fed back to the control chip to turn off the switch, so as to switch off the power supply circuit. After the temperature control chip monitors that the temperature of the device drops to 40° C., information about the temperature is fed back to the control chip to turn on the switch to restore the power supply of the power supply circuit, which makes the indicator lamp flash to prompt continued use.

According to the device provided by the present disclosure, preferably, the device further includes a main board, a metal substrate, a light source, a heat dissipation plate, a heat dissipation cover, a first cooling fin, and a second cooling fin.

The main board is provided with a control chip, and a control circuit.

The metal substrate is provided with a light source mounting surface, the light source is arranged on the light source mounting surface, and the metal substrate is electrically connected to the main board.

The heat dissipation plate is in contact with a surface, opposite to the light source mounting surface, of the metal substrate.

The heat dissipation cover is in contact with the light source mounting surface.

The first cooling fin and the second cooling fin are arranged on two opposite sides of the main board, respectively.

According to the device of the present disclosure, preferably, the device further includes a working sleeve, and the housing includes a lower housing, and a light source sleeve.

The metal substrate is provided with an elastic sheet, and the elastic sheet is provided to control the main board to connect to and disconnect from the light source.

The heat dissipation cover is provided with an elastic sheet opening, at least a part of a projection of the elastic sheet opening on a plane where the light source mounting surface coincides with a projection of the elastic sheet on the plane where the light source mounting surface.

The light source sleeve is sleeved outside the metal substrate, the heat dissipation plate and the heat dissipation cover; the light source sleeve is provided with a light source hole, and an elastic sheet button. The light source hole is provided for the transmission of light generated by the light source, and the elastic sheet button is arranged to touch the elastic sheet under the action of an external force.

The working sleeve is provided with a connector, and the connector is provided with a connector light-transmitting hole. The connector light-transmitting hole is provided for the transmission of light generated by the light source. When the working sleeve is sleeved on the periphery of the light source sleeve, the working sleeve touches the elastic sheet button.

The main board, the first cooling fin, the second cooling fin and the battery are arranged in the lower housing.

An oral hygiene system is also provided by the present disclosure, including the device above and a compound shown in Formula (I):

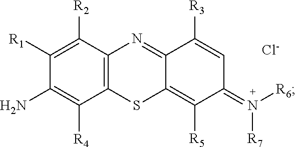

where R₁, R₂, R₃, R₄, R₅, R₆ and R₇ are independently selected from H or C1-C6 alkyl, respectively.

A use method of the oral hygiene device is also provided by the present disclosure, including the following steps: irradiating tissue in an oral cavity using red light and/or blue light.

An LED light source is used for replacing a semiconductor light source. The LED light source is low in cost and low in power, thus reducing the damage to the tooth by the heat generated by the light source. In addition, LED (630 nm) and semiconductor laser (810 nm) are used to irradiate a single-root extracted tooth infected with Enterococcus faecalis. The results show that the LED (630 nm) is more effective than the semiconductor laser (810 nm) in killing Enterococcus faecalis.

According to the preferable technical solution of the present disclosure, a cone-shaped disposable root canal light guide needle is mounted at the head of the device main body for converging an output light beam, thus reducing light spot area, making the root canal sterilization operation more convenient and sensitive, and reducing the possibility of secondary pollution.

Photodynamic therapy is introduced to treat oral diseases, so as to solve the defects of existing clinical techniques.

According to the preferable technical solution of the present disclosure, the LED (630 nm) and the semiconductor laser (810 nm) are used to irradiate dental caries plaque infected by Actinobacillus actinomycetemcomitans. The results show that the LED (630 nm) is more effective than the semiconductor laser (810 nm) in killing Actinobacillus actinomycetemcomitans.

According to the preferable technical solution of the present disclosure, a cylindrical disposable periodontal light guide needle is mounted at the head of the device main body for converging an output light beam, thus reducing light spot area, making the periodontal sterilization operation more convenient and sensitive, and reducing the possibility of secondary pollution.

According to the preferable technical solution of the present disclosure, the LED (630 nm) and the semiconductor laser (810 nm) are used to irradiate Porphyromonas gingivalis. The results show that the LED (630 nm) is more effective than the semiconductor laser (810 nm) in killing Porphyromonas gingivalis.

According to the preferable technical solution of the present disclosure, a disposable pure flat light guide needle is mounted at the head of the device main body for converging an output light beam to ensure that the light irradiates the lesion, thus making the periodontal and periapical sterilization operation more convenient and sensitive, and reducing the possibility of secondary pollution.

According to the preferable technical solution of the present disclosure, the common photodynamic therapy device is only suitable for small-area treatment, while the pure flat light guide needle above employs a method of directly performing photodynamic therapy close to the lesion, and thus can achieve large-area treatment. That is, the treatment requirements for large-area lesions in the oral cavity such as periapical periodontitis or periodontitis can be satisfied at the same time. The two diseases can be treated in the same way for one time, and the burden of patients can be reduced.

According to the preferable technical solution of the present disclosure, compared with the cone-shaped root canal light guide needle, the pure flat light guide needle is more suitable for the treatment of the large-area lesion, and it is necessary to expose the lesion properly in actual operation. For example, it is necessary to open the gingiva when treating the periapical periodontitis, while in the treatment of periodontitis, it is necessary to properly spread the periodontal pocket with tweezers to ensure the sufficient irradiation of the light.

According to the preferable technical solution of the present disclosure, by adding the rotatable power attenuation sheet, the attenuation sheet can be rotated to other corresponding positions by different replacement heads, thus effectively controlling the optical power and satisfying the convenience requirement of reuse only by replacing the light guide needle.

According to the preferable technical solution of the present disclosure, the rapid switching of methods and devices for treating periodontitis, root canal inflammation, peri-implantitis and dental periapical periodontitis can be achieved without losing convenience.

By simplifying the operation, the probability of secondary injury caused by human factors during treatment is reduced, which is beneficial to the patients.

According to the preferable technical solution of the present disclosure, the device is provided with a heat dissipation plate, a heat dissipation cover, a first cooling fin, and a second cooling fin, and thus heat energy generated by the light source can be dissipated in time, and the safety of the device is improved.

According to the preferable technical solution of the present disclosure, the device is provided with an elastic sheet and a supporting button thereof. Therefore, when the working sleeve is not connected, the light source cannot be turned on, thus preventing the human eye from being damaged by turning on the light source by mistake. The cooperation of the device provided by the present disclosure can play a good role in sterilization and anti-inflammation when cooperated with a specific compound, and has less damage to the tooth and tissue around the lesion.

### BRIEF DESCRIPTION OF THE DRAWINGS

FIG. 1 is a structural schematic diagram of an oral hygiene device according to the present disclosure;

FIG. 2 is an exploded view of an oral hygiene device in FIG. 1;

FIG. 3 is a schematic diagram of a head perspective structure of an oral hygiene device according to the present disclosure;

FIG. 4 is a schematic diagram of a head connecting structure of an oral hygiene device according to the present disclosure;

FIG. 5 is a structural schematic diagram of a root canal light guide needle of an oral hygiene device according to the present disclosure;

FIG. 6 is a schematic operation diagram of a device according to the present disclosure for post-root canal treatment;

FIG. 7 is a structural schematic diagram of an oral hygiene device according to the present disclosure;

FIG. 8 is an exploded view of an oral hygiene device shown in FIG. 7;

FIG. 9 is a perspective schematic diagram of a head structure assembled with a long periodontal light guide needle;

FIG. 10 is a perspective schematic diagram of a head structure assembled with a short periodontal light guide needle;

FIG. 11 is a local side view of FIG. 10;

FIG. 12 is a schematic operation diagram of a device according to the present disclosure for dental caries plaque cleaning or periodontitis sterilization;

FIG. 13 is a structural schematic diagram of an oral hygiene device according to the present disclosure;

FIG. 14 is an exploded view of an oral hygiene device shown in FIG. 13;

FIG. 15 is a perspective schematic diagram of a head structure assembled with a pure flat light guide needle;

FIG. 16 is a local side view of FIG. 15;

FIG. 17 is a schematic diagram of clinical operation of an oral hygiene device according to the present disclosure;

FIG. 18 is a structural schematic diagram of an oral hygiene device according to the present disclosure;

FIG. 19 is a schematic diagram of a tail structure of a connector according to the present disclosure;

FIG. 20 is a structural schematic diagram of a periodontal light guide needle;

FIG. 21 is a structural schematic diagram of a root canal light guide needle;

FIG. 22 is a structural schematic diagram of a pure flat light guide needle;

FIG. 23 is a rear view of a periodontal light guide needle shown in FIG. 20;

FIG. 24 is a rear view of a root canal light guide needle shown in FIG. 21;

FIG. 25 is a rear view of a pure flat light guide needle shown in FIG. 22;

FIG. 26 is an exploded view of an oral hygiene device shown in FIG. 18;

FIG. 27 is a structural schematic diagram of a power attenuation sheet;

FIG. 28 is a structural schematic diagram of a power attenuation sheet when completely shading a light source;

FIG. 29 is a structural schematic diagram of a power attenuation sheet when minimally shading a light source;

FIG. 30 is a structural schematic diagram of a power attenuation sheet when partially shading a light source;

FIG. 31 is a diagram of a power driving circuit;

FIG. 32 is a diagram of a constant-current source circuit;

FIG. 33 is a structural schematic diagram of an oral hygiene device according to the present disclosure;

FIG. 34 is an exploded view of a device shown in FIG. 33;

FIG. 35 is an enlarged view of a partial structure of a device shown in FIG. 34;

FIG. 36 is an exploded view of a local structure of a light guide needle and a working sleeve.

In the drawings:
1—charger; 2—housing; 4—battery; 21—connector; 211—clamping groove; 22—control chip; 221—temperature control chip; 222—current control chip; 23—light source; 24—working switch; 25—treatment time selection button; 26—light beam selection button; 27—control region; 3—root canal light guide needle; 31—mounting joint; 6—periodontal light guide needle; 7—control circuit; 9—pure flat light guide needle; 8—power attenuation sheet; 81—inner fixed sheet; 82—outer rotary sheet; 10—constant-current source circuit;
11—main board; 12—metal substrate; 1202—first metal substrate connecting hole; 1203—second metal substrate connecting hole; 1204—elastic sheet; 13—heat dissipation plate; 1301—heat dissipation plate body; 13011—first heat dissipation plate connecting hole; 13012—heat dissipation plate connecting pillar; 1302—heat dissipation plate connecting part; 13021—second heat dissipation plate connecting hole; 14—heat dissipation cover; 1401—heat dissipation cover body; 14011—heat dissipation cover connecting hole; 14012—elastic sheet opening; 1402—heat dissipation cover connecting part; 15—first cooling fin; 1501—first cooling fin connecting part; 15011—first cooling fin connecting hole; 1502—first cooling fin main board accommodating part; 16—second cooling fin; 1601—second cooling fin connecting part; 16011—second cooling fin connecting hole; 1602—second cooling fin main board accommodating part; 17—light source sleeve; 1701—light source hole; 1702—elastic sheet button; 18—working sleeve; 1801—connector light-transmitting hole; 110—charging interface 111—lower housing; 112—control button; 1131—first cavity part; 1132—second cavity part; 1133—third cavity part; 114—light guide needle.

### DETAILED DESCRIPTION OF THE EMBODIMENTS

In the present disclosure, a “housing” may also be called a “shell”, a “battery” may also be called a “battery pack”, a “light guide needle” may also be called a “light guide head”, a “light guide working tip”, or a “working tip”, a “pure flat light guide needle” may also be called a “blunt flat light guide head”, and a “light beam selection button” may also be called a “light source wavelength selection button”.

### <Oral Hygiene Device>

The oral hygiene device can be used for root canal sterilization disinfection or whitening, dental caries plaque cleaning or periodontitis sterilization, and peri-implantitis and periapical periodontitis elimination. Therefore, the oral hygiene device may also be called “a device for root canal sterilization disinfection or whitening”, “a device for dental caries plaque cleaning or periodontitis sterilization”, or a “device for peri-implantitis and periapical periodontitis elimination”. In some embodiments, the oral hygiene device is provided with a constant-current source circuit, and thus the oral hygiene device may also be called “an optical power constant-current source control device for oral treatment”.

The oral hygiene device includes a housing, a control chip, a light source, and a light guide needle.

The control chip is encapsulated in the housing to form a device main body. The control chip may be encapsulated at a handle inside the housing. In some embodiments, the light source is encapsulated in the housing to serve as a part of the device main body. The housing may be made of plastic. The light source may be an LED light source, which may also be called an “LED working light source”.

The light guide needle is mounted at the head of the housing using a mortise-tenon structure. In some embodiments, the head of the housing (a head structure of the housing) includes a connector. The connector may be concave. The light guide needle is mounted on the connector using the mortise-tenon structure. The light source is arranged on the connector, and the light source is electrically connected to the control chip and the battery. The light guide needle further includes a mounting joint. The mounting joint may be arranged at the bottom of the light guide needle. The light guide needle is in mortise-tenon connection and in interference fit with the connector through the mounting joint. The light source may be located at the center of the connector.

The housing may be provided with a working switch, a treatment time selection button, and a light beam selection button. The working switch, the treatment time selection button and the light beam selection button are all electrically connected to the control chip. The working switch, the treatment time selection button and the light beam selection button may be located on a working panel at the handle of the housing.

A timer may be arranged in the control chip, and a switch is linked to a power supply wire of the light source. When the light source is powered on, the timer starts to work, and when the timing reaches 2 min, the switch can be turned off immediately.

The oral hygiene device may also include a charging base.

The oral hygiene device may also include a battery. The battery may be encapsulated at the bottom of the housing.

The oral hygiene device may further include a charger. The battery is powered by the charger. The light source is powered by the battery. The charger provides an electrode to be electrically connected to the battery pack. The charger may be arranged at the bottom of the housing and embedded with the housing.

The device may include a constant-current source circuit. The constant-current source may be encapsulated in the housing. The rated output optical power of the constant-current source circuit is from 500 mW to 700 mW.

The device may include a control circuit. The control circuit is encapsulated in the housing.

In some embodiments, the oral hygiene device includes a charging base, a housing, a constant-current source circuit, a control chip, a light source, a light guide needle, and a control circuit.

The constant-current source circuit, the control chip and the control circuit are encapsulated in the housing. A hand-held section of the housing is provided with a control region including a working switch, a treatment time selection button, and a light velocity selection button, and the control region is electrically connected to the control chip.

The top of the housing is provided with a concave connector, the light source is arranged at the center of the connector, and is electrically connected to the working switch, the light velocity selection button and the control chip. The housing may be made of plastic, and may also be called a “plastic housing”.

In some embodiments, the connector is provided with four sections of clamping grooves, and the mounting joint is correspondingly provided with four sections of grooves. The clamping grooves are clamped into the grooves, and the connector is connected to and disconnected from the mounting joint in an interference manner.

In some embodiments, the connector is a concave connector. An edge of the connector is a circular arc. The edge of the connector is provided with at least two clamping grooves. The light guide needle is in mortise-tenon connection and in interference fit with the clamping groove through a corresponding convex mounting joint.

The rated optical power of the light source may be 25 mW or 0.5-1.5 W. In some embodiments, the rated optical power of the light source is 25 mW. In some embodiments, the rated optical power of the light source is 1 W. The light source may be a red-blue variable light source. The wavelength of the red light ranges from 630 nm to 650 nm. The wavelength of the blue light ranges from 430 nm to 450 nm. The light source may be a variable LED lamp bead. The light source may be hemispherical. The light source emits light beams around, and after the light source is connected to the light guide needle, some light beams are converged on a needle tip, and some light beams are lost in the propagation process. A lamp bead with the model of 3535RGB and the control chip can be employed.

The housing has a height of about 17 cm and has a weight of about 150 g. The housing is made of polyvinyl chloride, i.e., PVC.

The light guide needle may be made of bisphenol A polycarbonate. The bisphenol A polycarbonate may also be called bisphenol A carbonate, or bisphenol A PC.

The light guide needle may be a root canal light guide needle, a front end of the light guide needle is cone-shaped, and the optical power at a conical tip is 4.7 mW.

In some embodiments, a relationship between a device supply voltage U and output power P of the root canal light guide needle is as follows:

$$P = \frac{1}{( 0.26 + 1.38 {e}^{- U} )} ;$$

where e is a natural constant, a value of which is about 2.718281828 . . . .

The voltage of the device is provided by a battery or a battery pack, and the power supply voltage of the device is a battery or battery pack supply voltage.

A relationship between the output power P of the root canal light guide needle and the efficiency E of killing Enterococcus faecalis is as follows:

$$E = \frac{48}{1 + [ {( P / 2.58 )}^{- 2.37} ]} + 58.9 .$$

The light guide needle may be a periodontal light guide needle, and a front end of the periodontal light guide needle is cylindrical. The periodontal light guide needle has a length of 5 cm or 2 cm, and is used for converging the light source. The optical power of the cylindrical tip is 12.8 mW or 15.4 mW. Therefore, it is convenient for a photosensitizer entering the root canal of a tooth to play a sterilization role.

In some embodiments, a relationship between the device supply voltage U (V) and the output power P (W) of the periodontal light guide needle is as follows:

$$log ( P ) = 0.53 + 0.78 log ( U ) .$$

A voltage of the device is provided by the battery or battery pack, and the power supply voltage of the device is a battery or battery pack supply voltage.

The relationship between the output power P (W) of the periodontal light guide needle and the efficiency E (%) of killing Actinobacillus actinomycetemcomitans is as follows:

$$E = \frac{133}{1 + [ {( P / 10 )}^{- 1.5} ]} + 20.$$

The light guide needle may be a pure flat light guide needle. The pure flat light guide needle may also be called a “pure flat light guide working tip”. A front end of the pure flat light guide needle is in the shape of a frustum of a cone, the pure flat light guide needle has a length of 2 cm, and is used for irradiating the light source. The pure flat light guide needle may be used for irradiating lesions of periodontitis and periapical periodontitis.

After several illumination experiments, it is found that part of the light emitted by the light source will be scattered into the environment in actual use, resulting in loss. Therefore, the actual optical power of the pure flat light guide needle is from 12 mW to 16 mW, leading to a situation that the power of the light source is greater than the optical power of the light guide needle. The optical power of the pure flat light guide needle is from 12 mW to 16 mW, thus facilitating to directly irradiate a large-area lesion to control the photosensitizer to play a sterilization role.

In some embodiments, a relationship between the device supply voltage U (V) and the output power P (W) of the pure flat light guide needle is as follows:

$$log ( p ) = 0.45 + 0.83 log ( U ) .$$

A voltage of the device is provided by the battery or battery pack, and the power supply voltage of the device is a battery or battery pack supply voltage.

A relationship between the output power P (W) of the pure flat light guide needle and the efficiency E (%) of killing Porphyromonas gingivalis is as follows:

$$E = \frac{133}{1 + {( \frac{1}{p} )}^{3 / 2}} + 20.$$

A design principle of the pure flat light guide needle is as follows: in view of the need to sterilize and disinfect a large area in the actual treatment of periodontitis and periapical periodontitis, the tail of the light guide needle is designed as a smooth surface to meet the needs of refined treatment in the oral cavity and the need for a large range of light irradiation during the process. During actual use, the light emitted by the light source passes through the pure flat light guide needle, a small part of the light is scattered to the outside, and most of the light is emitted from a tail end of the light guide needle to finally irradiate on the photosensitizer, so as to cooperate with the photosensitizer to achieve sterilization and anti-inflammation operation for treating periodontitis and periapical periodontitis.

The innovative features and advantages of the pure flat light guide needle in technology are as follows: (1) According to the actual situation in the treatment of periodontitis and periapical periodontitis, the pure flat light guide needle employs the shape with relatively large volume and smooth tail end, so as to meet the need of large-scale sterilization and anti-inflammation in the treatment of the above oral diseases at one time. (2) The application range is wider in comparison with an existing oral photodynamic sterilization device, the large-area irradiation and refined operation can be both considered due to the reasonable design of the tail of the light guide needle, so as to achieve the simultaneous treatment of the periodontitis and periapical periodontitis. In practical use, it is only necessary to inject photosensitizer into both lesions and then irradiate the same.

In some embodiments, a relationship between the output power P of the light guide needle and the efficiency E of killing pathogenic bacteria such as Porphyromonas gingivalis is as follows:

$$E = {( \frac{1}{p} )}^{- 3 / 2} + 15.$$

A relationship between the output power P of the light guide needle and the efficiency E of killing Enterococcus faecalis is as follows:

$$E = \frac{35}{{( \frac{1}{p} )}^{\frac{7}{2}} - 1} .$$

The oral hygiene device may further include a power attenuation sheet. The power attenuation sheet is fixed to a front end of the light source. The power attenuation sheet is of a double-layer structure, and includes an inner fixed sheet and an outer rotary sheet in coaxial arrangement. Each of the inner fixed sheet and the outer rotary sheet is composed of six 30° fan-shaped blades rotating in an array at intervals of 30° from each other. The inner fixed sheet is fixed to the front end of the light source, the outer rotary sheet rotates around the center of the inner fixed sheet by 0-30°, and forms three different types of shading regions for the light source with the inner fixed sheet, which are complete shading, partial shading and minimum shading, and thus three different output power states are formed to achieve regulation of the optical power.

The light guide needle may be selected from one of a periodontal light guide needle, a root canal light guide needle, and a pure flat light guide needle. When using the periodontal light guide needle, the optical power transmitted by the light guide needle is from 60 mW to 85 mW, and the power attenuation sheet is in partial shading. When using the root canal light guide needle, the optical power transmitted by the light guide needle is from 40 mW to 50 mW, and the power attenuation sheet is in complete shading. When using the pure flat light guide needle, the optical power transmitted by the light guide needle is from 160 mW to 220 mW, and the power attenuation sheet is in minimum shading. The light guide needle is fixedly mounted on a connector.

The periodontal light guide needle is used for superficial treatment such as periodontitis and dental plaque irradiation. The root canal light guide needle is used for irradiation operation of dental pulp and root canal which need precise treatment. The pure flat light guide needle is used for operations of periapical periodontitis and peri-implantitis which require large-scale irradiation, sterilization and anti-inflammation.

The oral hygiene device may further include a current control chip. The current control chip is used for detecting the current in a power supply circuit. When the current is excessive, information about excessive current is fed back to the control chip to turn off the switch, so as to switch off the power supply circuit. Therefore, the battery is prevented from excessive life loss, and the service life of equipment is prolonged.

The oral hygiene device may further include a temperature control chip. The temperature control chip is linked with the indicator lamp through a switch. When the temperature control chip monitors that a temperature of the device is greater than 80° C., information about the temperature is fed back to the control chip to turn off the switch, so as to switch off the power supply circuit. After the temperature control chip monitors that the temperature of the device drops to 40° C., information about the temperature is fed back to the control chip to turn on the switch to restore the power supply of the power supply circuit, which makes the indicator lamp flash to prompt continued use. In some embodiments, the device further includes an indicator lamp.

In some embodiments, the power for treating dental caries plaque and periodontitis is between 11 mW and 16 mW. In some embodiments, the power for treating peri-implantitis and periapical periodontitis is between 12 mW and 16 mW. The 3535RGB lamp bead with the rated optical power of 25 mW and the control chip can be employed.

The oral hygiene device may further include a main board. The main board is provided to control the light source. The main board may control the lighting and extinguishing of the light source. The main board may control the lighting time of the light source. The main board may control a wavelength of the light emitted by the light source. According to one embodiment of the present disclosure, the main board may control the lighting and extinguishing of the light source, the lighting time of the light source, and the wavelength of the light emitted by the light source. The main board is provided with a control chip, and a control circuit. The control chip and the control circuit may employ conventional control chips and control circuits, and thus will not be described in detail here. The main board is electrically connected to the battery. The battery stores electric energy and supplies the electric energy to the main board.

The oral hygiene device may further include a metal substrate. The metal substrate is electrically connected to the main board. The metal substrate is provided with a first surface and a second surface opposite to each other, the first surface is a light source mounting surface, and a second surface is an opposite surface of the light source mounting surface. The light source mounting surface is used for mounting a light source (the first surface). The metal substrate may be provided with a first metal substrate connecting hole. The metal substrate, a heat dissipation plate and a heat dissipation cover can be connected through the first metal substrate connecting hole. The metal substrate may also be provided with a second metal substrate connecting hole. The second metal substrate connecting hole is used to fix a heat dissipation plate connecting pillar, thereby fixing opposite positions of the heat dissipation plate and the metal substrate. Specifically, the heat dissipation connecting pillar is located in the second metal substrate connecting hole. The metal substrate may be made of aluminum.

The metal substrate may be provided with an elastic sheet. The elastic sheet is used to control the main board to be connected to and disconnected from the light source. In some embodiments, the elastic sheet may be arranged to close to one end, close to the main board, of the metal substrate. The elastic sheet is arranged on the light source mounting surface.

The light source is arranged on the light source mounting surface. In some embodiments, the light source is arranged at one end, away from the main board, of the metal substrate. The light source may be an LED lamp source.

The oral hygiene device may further include a heat dissipation plate. The heat dissipation plate is provided to dissipate and conduct the heat generated by the light source. In some embodiments, the heat dissipation plate is provided to dissipate and conduct the heat generated by the light source to the first cooling fin. In some other embodiments, the heat dissipation plate is provided to dissipate and conduct the heat generated by the light source to the first cooling fin and the second cooling fin. The heat dissipation plate is in contact with a surface (second surface), opposite to the light source mounting surface, of the metal substrate. Preferably, the heat dissipation plate is attached to the surface, opposite to the light source mounting surface, of the metal substrate. According to one embodiment of the present disclosure, the heat dissipation plate is connected to, or in contact with, the heat dissipation cover. In some embodiments, the heat dissipation plate is further connected to, or in contact with, the first cooling fin. In some other embodiments, the heat dissipation plate is further connected to, or in contact with, the first cooling fin and the second cooling fin.

The heat dissipation plate may be provided with a heat dissipation plate body, and a heat dissipation plate connecting part. The heat dissipation plate body is connected to the heat dissipation plate connecting part. The heat dissipation plate body and the heat dissipation plate connecting part may be of an integrated structure.

The heat dissipation plate body is attached to a surface (second surface), opposite to the light source mounting surface, of the metal substrate. The area of a projection of the heat dissipation plate body on a plane where the light source mounting surface is located is greater than or equal to the area of the light source mounting surface. In some embodiments, the projection of the heat dissipation plate body on the plane where the light source mounting surface is located coincides with the light source mounting surface. The heat dissipation plate body may be provided with a first heat dissipation plate connecting hole. The heat dissipation plate can be connected to the metal substrate and the heat dissipation cover through the first heat dissipation plate connecting hole. The heat dissipation plate body may be provided with a heat dissipation plate connecting pillar. The heat dissipation plate connecting pillar is in fit with the second metal substrate connecting hole, thereby fixing opposite positions of the heat dissipation plate and the metal substrate. Specifically, the heat dissipation plate connecting pillar is located in the second metal substrate connecting hole.

In a direction perpendicular to the light source mounting surface, a thickness of the heat dissipation plate connecting part is greater than that of the heat dissipation plate body, thus making the heat dissipation plate connecting part form a bulge on one side where the metal substrate is located. The heat dissipation plate connecting part is provided with a second heat dissipation plate connecting hole. The second heat dissipation plate connecting hole is used for being connected to the first cooling fin and the second cooling fin. The heat dissipation plate connecting part may be provided with a groove. A connecting wire for connecting the main board to the metal substrate is arranged in the groove.

The metal substrate may be arranged on the bulge formed by the heat dissipation plate connecting part. In some embodiments, the metal substrate is abutted against with the bulge formed by the heat dissipation plate connecting part.

The heat dissipation plate is made of metal.

The heat dissipation plate can play a role of primary heat dissipation, and can transfer heat to the first cooling fin and the second cooling fin, thus rapidly reducing the heat generated by the light source. The heat dissipation plate is small in structure and compact in combination with other components, so as to extend into the oral cavity for treatment.

The oral hygiene device may further include a heat dissipation cover. The heat dissipation cover is provided to dissipate and conduct the heat generated by the light source. In some embodiments, the heat dissipation cover is provided to dissipate and conduct the heat generated by the light source to the second cooling fin. In some other embodiments, the heat dissipation cover is provided to dissipate and conduct the heat generated by the light source to the first cooling fin and the second cooling fin. The heat dissipation cover is in contact with the light source mounting surface (the first surface). In some embodiments, the light source mounting surface is covered with the heat dissipation cover, without shading the light source.

The heat dissipation cover may be provided with a heat dissipation cover body, and a heat dissipation cover connecting part. The heat dissipation cover body is connected to the heat dissipation cover connecting part. The heat dissipation cover body and the heat dissipation cover connecting part may be of an integrated structure.

The light source mounting surface is covered with the heat dissipation cover body. The heat dissipation cover body does not shade the light source. The area of a projection of the heat dissipation cover body on the plane where the light source mounting surface is located is greater than or equal to the area of the light source mounting surface. In some embodiments, the projection of the heat dissipation cover body on the plane where the light source mounting surface is located coincides with the light source mounting surface. An outer profile of the section of the heat dissipation cover body is a convex arc. The convex arc refers to bulging to a direction away from the metal substrate. Therefore, the surface area of the heat dissipation cover body can be increased, and the heat conduction and diffusion efficiency are improved. Such a heat dissipation cover body has small volume and is convenient for use.

The heat dissipation cover connecting part is connected to the heat dissipation plate connecting part. A conductive substrate, the heat dissipation plate and the heat dissipation cover are integrated together through a first conductive substrate connecting hole, a first heat dissipation plate connecting hole and a heat dissipation cover connecting hole. Specifically, a first fixing screw passes through the first conductive substrate connecting hole, the first heat dissipation plate connecting hole and the heat dissipation cover connecting hole, thereby connecting the conductive substrate, the heat dissipation plate and the heat dissipation cover together. Therefore, the conductive substrate is fixed between the heat dissipation cover and the heat dissipation plate.

The heat dissipation cover may be provided with an elastic sheet opening. The elastic sheet opening may be located on the heat dissipation cover body. A position of the elastic sheet opening is in fit with an elastic sheet. Specifically, at least a part of projection of the elastic sheet opening on the plane where the light source mounting surface is located coincides with a projection of the elastic sheet on the plane where the light source mounting surface is located. In some embodiments, the projection of the elastic sheet opening on the plane where the light source mounting surface is located falls within the range of the projection of the elastic sheet on the plane where the light source mounting surface is located. In some other embodiments, the projection of the elastic sheet opening on the plane where the light source mounting surface is located falls into the middle of the projection of the elastic sheet on the plane where the light source mounting surface is located.

The heat dissipation cover is made of metal.

In some other embodiments, the oral hygiene device provided by the present disclosure includes a first cooling fin, and a second cooling fin. The first cooling fin and the second cooling fin are located on two opposite sides of the main board, respectively. The first cooling fin is located on one side of the main board. The second cooling fin is located on the other side of the main board.

The surface area of the first cooling fin is greater than that of the heat dissipation plate. The surface area of the first cooling fin is greater than that of the heat dissipation cover. The first cooling fin is connected to the main board. The first cooling fin is in contact with the heat dissipation plate and/or the heat dissipation cover. In some embodiments, the first cooling fin is provided with a first cooling fin connecting part, and a first cooling fin main board accommodating part. The first cooling fin connecting part is connected to the first cooling fin main board accommodating part. The first cooling fin connecting part and the first cooling fin main board accommodating part may be of an integrated structure. The first cooling fin may be made of metal.

The surface area of the second cooling fin is greater than that of the heat dissipation plate. The surface area of the second cooling fin is greater than that of the heat dissipation cover. The second cooling fin is connected to the main board. The second cooling fin is in contact with the heat dissipation plate and/or the heat dissipation cover. In some embodiments, the second cooling fin is provided with a second cooling fin connecting part, and a second cooling fin main board accommodating part. The second cooling fin connecting part is connected to the second cooling fin main board accommodating part. The second cooling fin connecting part and the second cooling fin main board accommodating part may be of an integrated structure. The second cooling fin may be made of metal.

The first cooling fin connecting part is connected to the heat dissipation plate and/or the heat dissipation cover. In some embodiments, the first cooling fin connecting part is connected to the heat dissipation plate. The first cooling fin connecting part may be provided with a first cooling fin connecting hole.

The second cooling fin connecting part is connected to the heat dissipation plate and/or the heat dissipation cover. In some embodiments, the second cooling fin connecting part is connected to the heat dissipation plate. The second cooling fin connecting part may be provided with a second cooling fin connecting hole.

According to one embodiment of the present disclosure, the first cooling fin and the second cooling fin are located on both sides of the heat dissipation plate, respectively. A second fixing screw passes through the first cooling fin connecting hole, the second cooling fin connecting hole and the second heat dissipation connecting hole, so as to fix the first cooling fin, the second cooling fin and the heat dissipation plate.

The first cooling fin main board accommodating part forms a main board accommodating cavity with the second cooling fin main board accommodating part. The main board accommodating part of the first cooling fin is opposite to the main board accommodating part of the second cooling fin. The main board accommodating cavity can accommodate the main board. The main board may be fixedly connected to an upper main board accommodating part and a lower main board accommodating part.

The main board accommodating cavity is provided with a first cavity part, a second cavity part, and a third cavity part which are in communication in turn. The first cavity part is close to the first cooling fin connecting part and the second cooling fin connecting part. In the first cavity part, a distance between the first cooling fin and the second cooling fin is denoted by F. In the second cavity part, a distance between the first cooling fin and the second cooling fin is denoted by S. In the third cavity part, a distance between the first cooling fin and the second cooling fin is denoted by T, where T is less than S and is greater than or equal to F. In some embodiments, T is less than S and is greater than F. Therefore, the heat conduction efficiency can be improved. The first cooling fin located at the second cavity part bulges to a direction away from the main board. The second cooling fin located at the second cavity part bulges to a direction away from the main board. Therefore, the holding of a user is facilitated, the surface area of the heat dissipation cover body can be increased, and the heat conduction efficiency is improved.

The oral hygiene device may further include a light source sleeve. The light source sleeve is sleeved outside the metal substrate, the heat dissipation plate and the heat dissipation cover.

The light source sleeve is provided with a light source. Light generated by the light source can pass through the light source. The area of a projection of the light source hole on the plane where the light source mounting surface is located is greater than the area of a projection of the light source on the plane where the light source mounting surface is located. Preferably, the projection of the light source on the plane where the light source mounting surface is located falls within the range of the projection of the light source hole on the plane where the light source mounting surface is located. Preferably, the projection of the light source on the plane where the light source mounting surface is located falls into the middle of the projection the light source hole on the plane where the light source mounting surface is located.

The light source sleeve is provided with an elastic sheet button. The elastic sheet button is provided to touch the elastic sheet under the action of an external force. In some embodiments, at least partial elastic sheet is arranged on an outer surface of the light source sleeve and protrudes to an outside of the light source sleeve. The elastic sheet button can move to an elastic sheet direction under the action of the external force. A projection of the elastic sheet button on the plane where the light source mounting surface is located falls within the range of a projection of an elastic sheet opening on the plane where the light source mounting surface is located. The elastic sheet button touches the elastic sheet through the elastic sheet opening, thereby compressing the elastic sheet and making the main board communicate with the light source.

The housing may include a lower housing, and a working sleeve. The working sleeve is provided with a connector. The connector is provided with a connector light-transmitting hole. The connector light-transmitting hole is provided for the transmission of light generated by the light source. The connector is connected to the light guide needle. The light generated by the light source can pass through the connector light-transmitting hole. The area of a projection of the connector light-transmitting hole on the plane where the light source mounting surface is located is greater than or equal to the area of the projection of the light source on the plane where the light source mounting surface is located. When the working sleeve is sleeved on the periphery of the light source sleeve, the projection of the light source on the plane where the light source mounting surface is located falls within the range of the projection of the connector light-transmitting hole on the plane where the light source mounting surface is located. Due to the extrusion effect of the working sleeve on the elastic sheet button, the elastic sheet button moves to an elastic sheet direction to compress the elastic sheet, thus making the light source communicate with the main board.

The working sleeve may be connected to the lower housing. The connection mode between the working sleeve and the lower housing is not limited, and the conventional connection mode in the field can be adopted, such as snap connection or tenon-mortise connection.

The oral hygiene device may further include a charging interface. The charging interface supplies electric energy to the battery. The charging interface is provided with a power supply connecting terminal, and the power supply connecting terminal is connected to an external power supply or the charger.

In some embodiments, the battery, the main board, the first cooling fin, the second cooling fin and at least part of the charging interface are located in a cavity formed by the lower housing. The lower housing is provided with a housing opening, through which the power supply connecting terminal is exposed outside.

### <Oral Hygiene System>

An oral hygiene system provided by the present disclosure includes the oral hygiene device above and a compound shown in Formula (I):

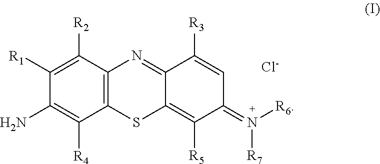

The oral hygiene device is specifically described as above, and thus will not be described in detail here.

In Formula (I), R₁, R₂, R₃, R₄, R₅, R₆ and R₇ are independently selected from H or C1-C6 alkyl, respectively. Preferably, R₁, R₂, R₃, R₄, R₅, R₆ and R₇ are independently selected from H or C1-C3 alkyl, respectively. Examples of C1-C6 alkyl groups include, but are not limited to, methyl, ethyl, n-propyl, isopropyl, n-butyl, isobutyl, tert-butyl, n-pentyl, isopentyl, neopentyl, n-hexyl and the like. In some embodiments, R₁, R₆ and R₇ are respectively methyl, and R₂, R₃, R₄ and R₅ are respectively H.

According to one embodiment of the present disclosure, the compound has the structure as shown in the following:

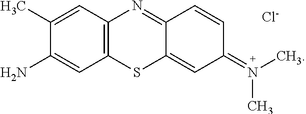

The compound, when cooperating with the device provided by the present disclosure may achieve better sterilization and anti-inflammation effect.

### <Use Method of Oral Hygiene Device>

The oral hygiene device can be used for dental caries plaque cleaning or periodontitis sterilization, root canal sterilization disinfection or whitening, and peri-implantitis and periapical periodontitis elimination. Therefore, a use method of the oral hygiene device may also be called “a method for dental caries plaque cleaning or periodontitis sterilization”, “a method for root canal sterilization disinfection or whitening”, or “a method for peri-implantitis and periapical periodontitis elimination”. In addition, the use method may also be called an optical power constant-current source control method for oral treatment.

The use method of the oral hygiene device provided by the present disclosure includes the following steps: irradiating teeth or tissue in the oral cavity using red light and/or blue light. Preferably, toluidine blue O is found on teeth or tissue in the oral cavity irradiated by red light and/or blue light.

The method above may specifically include the following steps: Step 1. In aerobic environment, a toluidine blue O photosensitizer is attached to bacteria, and the photosensitizer is in a singlet state at this time. Step 2. After being irradiated by an external light source, the photosensitizer is excited to transition to an excited singlet state, and then quickly transition to an excited triplet state. Step 3. The triplet state of the photosensitizer directly collides with tissue oxygen to release energy and return to the singlet state, and the released energy is absorbed by tissue oxygen to generate singlet reactive oxygen. Step 4. Reactive oxygen species produce rapid lipid oxidation on the bacteria, which affects the metabolism of the bacteria, and leads to rapid death of the bacteria, thus achieving sterilization and anti-inflammatory effects.

In the following embodiments, the light source may be an “LED light source”, or an “LED working light source”.

In some embodiments, the method further includes an indicator lamp steps:
Step 1. When treating a root canal clinically, normal saline is injected into the root canal for rinsing, then a root canal light guide needle is taken out and mounted on the head of a housing.
Step 2. A working switch button on a device housing is pressed for 3 s to start the device. A light beam selection button on the device housing is pressed until an upper indicator lamp shows red, indicating that a selected light beam wavelength is red light of 630 nm. A treatment time selection button on the device housing is pressed until a lower indicator lamp shows white, indicating that the working time of a light source is 10 s. A root canal light guide needle is close to the root canal of a tooth, the working switch button is pressed to start a light source for photodynamic sterilization. After 10 s, the sterilization process is completed, the light source is turned off automatically. The light beam selection button on the device housing is pressed until the upper indicator lamp shows blue, indicating that a selected light beam wavelength is blue light of 450 nm, and the root canal is irradiated again for 10 s.
Step 3. Step two is repeated for three times, the device is taken out of the oral cavity after the light source is automatically turned off, the working switch button on the device housing is pressed for 3 s to turn off the device, and finally, the inherent shape and function of the tooth are restored by filling a material or using other specific ways.

In the present disclosure, the root canal light guide needle outputs the red light as auxiliary light, which cooperates with the photosensitizer toluidine blue O (with a concentration of 0.1 mg/ml) for photodynamic therapy, thereby killing the bacteria in the tooth root canal.

The working switch is used for controlling the on and off of the device, and controlling the on and off of the light source.

The treatment time selection button is used for controlling the working time of the device. The working time may be 10 s, or 30 s. When the working time is 10 s, the device is mainly used for post-operative root canal therapy. When the working duration is 30 s, the device is mainly used for periodontal bacteria killing.

The light beam selection button is used for controlling a wavelength of the light source. The light source may be red light with a wavelength of 650 nm or blue light with a wavelength of 450 nm. The red light with the wavelength of 650 nm is mainly used for sterilization and disinfection after root canal therapy. The blue light with a wavelength of 450 nm is mainly used for whitening and cold light of the tooth.

The red light is used for sterilization after root canal therapy: after root canal resection of the tooth in clinic, normal saline is injected into the post-operative cavity for rinsing, and then a disposable root canal light guide needle is taken out and mounted at the head of the device. The red light of the device is selected as the auxiliary light, and 0.1 mg/ml toluidine blue O photosensitizer is injected into the post-operative cavity with a syringe. The root canal light guide needle deeps into a coronal access cavity, the light source is started for photodynamic sterilization for 10 s. The device is taken out from the oral cavity after the light source is automatically turned off. Finally, the inherent shape and function of the tooth are restored by filling a material or using other specific ways.

The blue light is used for tooth whitening: when the tooth is whitened in clinic, the normal saline is first used to clean the tooth, and a whitening agent light-curing resin (which may also be called light-curing resin) is applied to the tooth surface evenly, the blue light is selected as the auxiliary light, the device is close to the tooth surface, the light source is started to irradiate the tooth for 30 s, and then stands for 10 s. Such operations are repeated for three times, the device is taken out of the oral after the light source is automatically turned off, thus completing the tooth whitening.

The oxidation-reduction effect (which can be simply understood as bleaching effect) produced by peroxide in the whitening agent is used for tooth whitening. Working under the irradiation of cold light (blue light) is mainly to isolate ultraviolet rays and infrared rays, help the whitening agent penetrate into teeth for decoloration, and strengthen the effect of tooth whitening.

A fuse mechanism is arranged in the control chip of the device, which is triggered after the light source continuously works for 2 min to make the LED working light source automatically stop, so as to prevent irreversible damage to gingiva caused by excessive red and blue light power and long-term irradiation.

In some embodiments, the method further includes the following steps:
Step 1. Starting: normal saline is first injected into caries plaque for rinsing, and a device is removed from a charger, and then a disposable periodontal light guide needle is taken out and mounted at the head of the device. A long periodontal light guide needle and a short periodontal light guide needle are provided, the short periodontal light guide needle is used for external periodontal and the long periodontal light guide needle is used for internal periodontal. A working switch button on the housing is pressed for 3 s to start the device. The short periodontal light guide needle has a length of 2 cm, and the long periodontal light guide needle has a length of 5 cm.
Step 2. For superficial caries:
S2.1. A light beam selection button on the housing is pressed until an upper indicator lamp shows red, indicating that a selected light beam wavelength is red light. A treatment time selection button on the housing is pressed until a lower indicator lamp shows white, indicating that the working time of the light source is 10 s. The periodontal light guide needle is close to the caries plaque, then the working switch button is pressed to start the light source for photodynamic sterilization, and the sterilization process is completed after 10 s. The light source is turned off automatically, the light beam selection button on the housing is pressed until the upper indicator lamp shows blue, indicating that the selected light beam wavelength is blue light, and the caries plaque is irradiated again for 10 s.
S2.2. Above operation is repeated for three times, the device is taken out from the oral cavity after the light source is normally turned off, the working switch button on the housing is pressed for 3 s to turn off the device, and finally, the inherent shape and function of the tooth are restored by filling a material or using other specific ways, thus completing the treatment of superficial caries.
Step 3. For deep caries:
S3.1. The light beam selection button on the housing is pressed until the upper indicator lamp shows red, indicating that the selected light beam wavelength is red light. The treatment time selection button on the housing is pressed until the lower indicator lamp shows yellow, indicating that the working time of the light source is 30 s. afterwards, 0.1 mg/ml toluidine blue O photosensitizer is injected into a deep caries cavity with a syringe.
S3.2. The periodontal light guide needle deeps into the deep caries cavity, the working switch button is pressed to start the light source for photodynamic sterilization, and the sterilization process is completed after 30 s. The light source is turned off normally, the device is taken out from the oral cavity, the working switch button on the housing is pressed for 3 seconds to turn off the device, and finally, the inherent shape and function of the tooth are restored by filling a material or using other specific ways, thus completing the treatment of deep caries.
Step 4. For periodontitis:

The operation is consistent with the operation for deep caries, the toluidine blue O photosensitizer is applied to the lesion, and then the red light is used for photodynamic therapy.
Step 5. If the light source continues to work for 2 min due to an unexpected situation in Step 2 or Step 3, a fuse mechanism of a control chip is triggered, which makes the LED working light source stop automatically, thus preventing irreversible damage to gingiva caused by excessive red and blue light power and long-term irradiation.

In some embodiments, the method further includes an indicator lamp steps:
Step 1. Symptom identifying
S1.1. When a patient suffers from periapical periodontitis, the periapical periodontitis is treated according to Step 2.
S1.2. When a patient suffers from periodontitis, the periodontitis is treated according to Step 3.
S1.3. When a patient suffers from both periapical periodontitis and periodontitis, the periapical periodontitis and periodontitis are treated in the order of Step 2 and Step 3.
Step 2. For periapical periodontitis
S2.1. Pre-operation. A lesion is fully exposed after the incision of gingiva at a corresponding site, and a drainage operation is carried out to remove pus, blood, etc. on the surface of the lesion, and then the lesion site is rinsed with normal saline.
S2.2. Starting. The device is removed from a charger, a disposable pure flat light guide needle is taken out and mounted on the head of the device, where the pure flat light guide needle is a replaceable device shaped in the shape of a frustum of a cone and can act on the surface of the lesion after incision. The working switch button on the housing is pressed for 3 s to start the device.
S2.3. The light beam selection button on the housing is pressed until the upper indicator lamp shows red, indicating that the selected light beam wavelength is red light. The treatment time selection button on the housing is pressed until the lower indicator lamp shows yellow, indicating that the working time of the light source is 30 s. Afterwards, the 0.1 mg/ml toluidine blue O photosensitizer is injected into the incision using a needle-free syringe.
S2.4. The pure flat light guide needle is placed to close to the lesion, the working switch button is pressed to start the light source for photodynamic sterilization, and the sterilization process is completed after 30 s. The light source is turned off normally, the device is taken out from the oral cavity, the working switch button on the housing is pressed for 3 s to stop power supply, and then the lesion incision on the gingiva is closed, and disinfected, assisted by corresponding analgesic drugs.
Step 3. For periodontitis
S3.1. Pus is sucked, and a lesion region is rinsed with normal saline.
S3.2: Starting. The device is removed from the charger, and a disposable pure flat light guide needle is taken out and mounted on the head of the device, where the pure flat light guide needle is a replaceable device shaped in the shape of a frustum of a cone and can act on the surface of the lesion after incision. The working switch button on the housing is pressed for 3 s to start the device.
S3.3. The light beam selection button on the housing is pressed until the upper indicator lamp shows red, indicating that the selected light beam wavelength is red light. The treatment time selection button on the housing is pressed until the lower indicator lamp shows yellow, indicating that the working time of the light source is 30 s. Afterwards, 0.1 mg/ml toluidine blue O photosensitizer is injected into a periodontal pocket using a needle-free syringe.
S3.4. The periodontal pocket is slightly spread using tweezers, the pure flat light guide needle is close to the photosensitizer as much as possible. The working switch is pressed to start the light source for photodynamic sterilization, and the sterilization process is completed after 30 s. The light source is turned off normally, the device is taken out from the oral cavity, and the working switch button on the housing is pressed for 3 s to stop the power supply.
S3.5. If the light source continues to work for 2 min due to an unexpected situation, the fuse mechanism of the control chip is triggered, which makes the LED working light source stop automatically, thus preventing irreversible damage to gingiva caused by excessive red and blue light power and long-term irradiation.

In some embodiments, the method further includes the following steps:
Method 1. Sterilization operation for periodontitis, dental plaque and caries is as follows:
S1.1. An operation space is rinsed, and then the water in the oral cavity is dried as much as possible with biological cotton.
S1.2. A short periodontal light guide needle is mounted as the light guide head, the working switch on the housing is pressed for 1 s until the indicator lamp flashes alternately in red and blue, then the light source wavelength selection button is pressed to select a light source, where the red light is used for superficial anti-inflammation and dental plaque, and it is indicated that the system is ready when observing that the indicator lamp turns red and always lights up.
S1.3. The photosensitizer toluidine blue O (TBO for short) is applied to the periodontal surface, the periodontal light guide needle extends into the oral cavity, then the working switch is pressed again to make the system start to work, where the single irradiation lasts for 30 seconds, and the periodontal light guide needle rotates around a tooth lesion for irradiation during the process. After cleaning, the photosensitizer in the oral cavity is cleaned with normal saline until there is no color attached.
S1.4. After the sterilization process is completed after 30 s, the light source is turned off normally, the device is taken out from the oral cavity, and the working switch button on the housing is pressed for 1 s to stop the power supply.
Method 2. Sterilization operation in dental pulp and root canal is as follows:
S2.1. An operation space is rinsed, and then the water in the oral cavity is dried as much as possible with biological cotton.
S2.2. The light guide needle is replaced with a long periodontal light guide needle, the working switch on the housing is pressed for 1 s until the indicator lamp flashes alternately in red and blue, then the light source wavelength selection button is pressed to select the light source. For normal root canal filling and cleaning, if the patient does not have special circumstances such as the lesion, the lesion is directly irradiated with the blue light, without using toluidine blue O for cooperation, and it is indicated that the system is ready when observing that the indicator lamp turns blue and always lights up.
S2.3. The photosensitizer toluidine blue O (TBO for short) is injected into the root canal with a needle tubing. For deep root canal, liquid bubbles are produced using a root canal scrubber after the injection, then the root canal light guide needle extends into the oral cavity to align with the root canal of the tooth, the working switch is pressed again to make the system start to work, where the duration of single irradiation is 60 s, and an inflammatory site should be irradiated emphatically during the process. After cleaning, the root canal is cleaned with the normal saline until there is no color attached.
S2.4. After the sterilization process is completed after 60 s, the light source is turned off normally, the device is taken out from the oral cavity, and the working switch button on the housing is pressed for 1 s to stop the power supply.
Method 3. Sterilization operation for peri-implantitis is as follows:
S3.1. The pus is sucked, and a lesion region is rinsed with normal saline.
S3.2. The light guide needle is replaced with a pure flat light guide needle, the working switch on the housing is pressed for 1 s until the indicator lamp flashes alternately in red and blue, and then the light source wavelength selection button is pressed to select a light source. The red light should be used for irradiating the lesion for the peri-implantitis, the photosensitizer toluidine blue O is applied to the lesion site of the peri-implantitis, the photosensitizer toluidine blue O is injected into a gingival surface with a syringe, the light guide needle extends into the oral cavity to align with the lesion of the peri-implantitis, and then the working switch is pressed again, where the duration of single irradiation is 90 s. An inflammatory site should be irradiated emphatically during the process. After cleaning, the photosensitizer in the oral cavity is cleaned with normal saline until there is no color attached.
S3.3. After the sterilization process is completed after 90 s, the light source is turned off normally, the device is taken out from the oral cavity, and the working switch button on the housing is pressed for 1 s to stop the power supply.
Method 4. Sterilization operation for dental periapical periodontitis is as follows:
S4.1. A lesion is localized, the pus is sucked, and a lesion region is rinsed with normal saline.
S4.2. The pure flat light guide needle is mounted as the light guide needle, the working switch on the housing is pressed for 1 s until the indicator lamp flashes alternately in red and blue, and then the light source wavelength selection button is pressed to select a light source, where the red light is used for irradiating a lesion for dental periapical periodontitis. The photosensitizer toluidine blue O is applied to a lesion site of the gingival surface, the light guide head extends into the oral cavity to align with the lesion of the periodontitis, and then the working switch is pressed again to make the system start to work, where the duration of single irradiation is 90 s. The incision site is emphatically irradiated during the process. After cleaning, the photosensitizer in the oral cavity is cleaned with normal saline until there is no color attached.
S4.3. After the sterilization process is completed after 90 s, the light source is turned off normally, the device is taken out from the oral cavity, and the working switch button on the housing is pressed for 1 s to stop the power supply.

Particularly, when the light source is powered on, the timer starts to work, it is specified that the working temperature of the device is not higher than 80° C. Once the temperature is excessively high, the switch is off under the control of the temperature control chip, the power supply circuit is turned off, and the cooling is started. When the temperature drops to 40° C., the indicator lamp on the device flashes to indicate the continued use.

Particularly, when the patient suffers from diseases such as oral fibrosis, further stimulation to oral mucosa should be avoided. All the above operations are carried out with red light.

### Embodiment 1

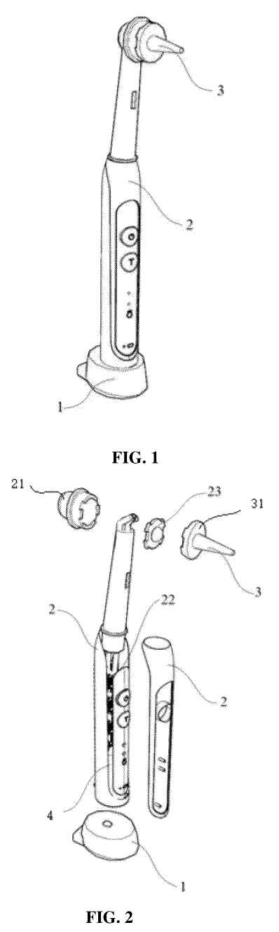

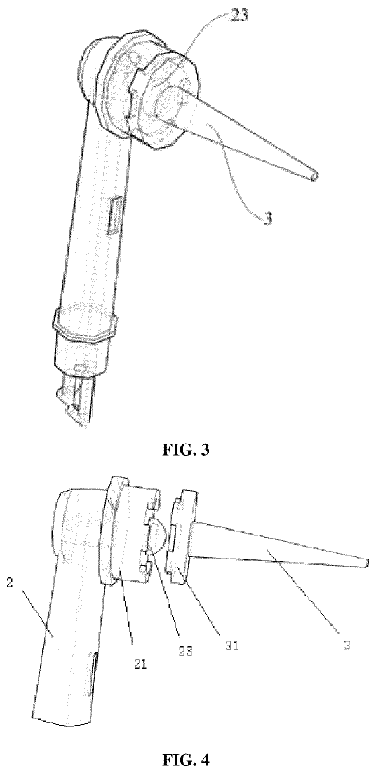

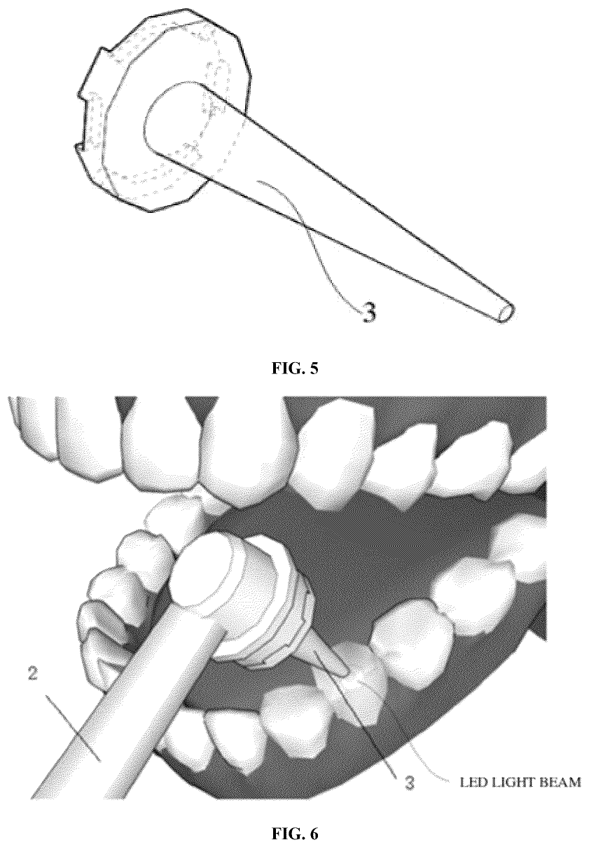

As shown in FIG. 1 to FIG. 6, a device for root canal sterilization and disinfection or whitening provided by the present disclosure includes a housing 2, a battery 4, a control chip 22, a light source 23, and a root canal light guide needle 3.

The battery 4, the control chip 22 and the light source 23 are encapsulated in the housing 2 to form a device main body. The housing 2 is further provided with a working switch 24, a treatment time selection button 25, and a light beam selection button 26. The working switch 24, the treatment time selection button 25 and the light beam selection button 26 are all electrically connected to the control chip 22.

The root canal light guide needle 3 is mounted at the head of the housing 2 using a mortise-tenon structure.

A head structure of the housing 2 includes a connector 21, the light source 23 is arranged at the center of the connector 21, and the light source 23 is electrically connected to the control chip 22 and the battery 4. The root canal light guide needle 3 further includes a mounting joint 31, which is in mortise-tenon connection and in interference fit with the connector 21 through the mounting joint 31.

The connector 21 is provided with four sections of clamping grooves, and the mounting joint 31 is correspondingly provided with four sections of grooves, and the clamping grooves are clamped into the grooves, and the connector 21 is connected to or disconnected from the mounting joint 31 in an interference manner.

The device further includes a charger 1, the battery 4 is powered by the charger 1, and the light source 23 is powered by the battery 4. The rated optical power of the light source 23 is 25 mW. The light source 23 is a red-blue variable light source, where a wavelength of the red light ranges from 630 nm to 650 nm, and a wavelength of the blue light ranges from 430 nm to 450 nm. A 3535RGB lamp bead and a control chip are generally employed.

The root canal light guide needle 3 is made of bisphenol A carbonate (PC for short), a front end of the root canal light guide needle is cone-shaped, the root canal light guide needle has a length of 5 cm, is used for converging the light source, and the optical power of a conical tip is 4.7 mW. Therefore, it is convenient for the photosensitizer entering the root canal of tooth to play a sterilization role.

The housing 2 has a height of about 17 cm, and a weight of about 150 g, and is made of polyvinyl chloride (PVC).

### Embodiment 2

After the device in Embodiment 1 is used after root canal resection of a tooth in clinic, the normal saline is injected into a postoperative cavity for rinsing. The device is removed from the charger, then a disposable root canal light guide needle is taken out and mounted on the head of the device, a working switch on the device housing is pressed for 3 s to start the device. A beam selection button on the housing is pressed until an upper indicator lamp shows red, indicating that the selected light beam wavelength is red light of 630 nm. A treatment time selection button on the device housing is pressed until a lower indicator lamp shows white, indicating that the working time of the light source is 10 s. afterwards, 0.1 mg/ml toluidine blue O photosensitizer is injected into the postoperative cavity with a syringe. The root canal light guide needle deeps into a coronal access cavity, the working switch is pressed to start the light source for photodynamic sterilization. After 10 s, the sterilization process is completed, and the light source is automatically turned off. The device is taken out from the oral cavity. The working switch 3 on the device housing is pressed for 3 s to turn off the device. Finally, the filling material is combined with the root canal dentin to complete the root canal therapy.

### Embodiment 3

The photodynamic sterilization experiment on Enterococcus faecalis ATCC2921 is carried out using the device provided by the present disclosure and the photosensitizer toluidine blue O, and a red-light beam with a wavelength of 630 nm is selected to irradiate for 30 s. The experimental results show that the sterilization effect of the tip of the root canal light guide needle is obvious, and the sterilization efficiency can reach 99%.

### Embodiment 4

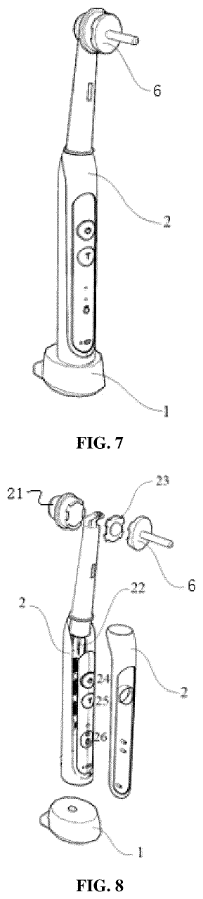

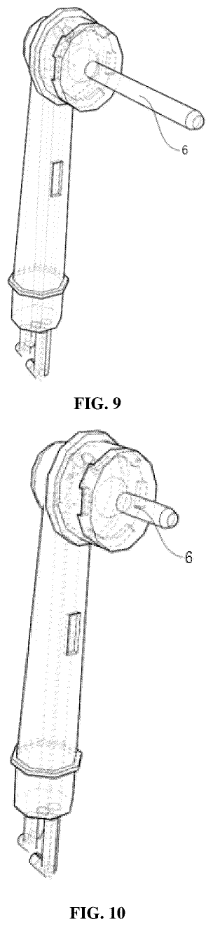

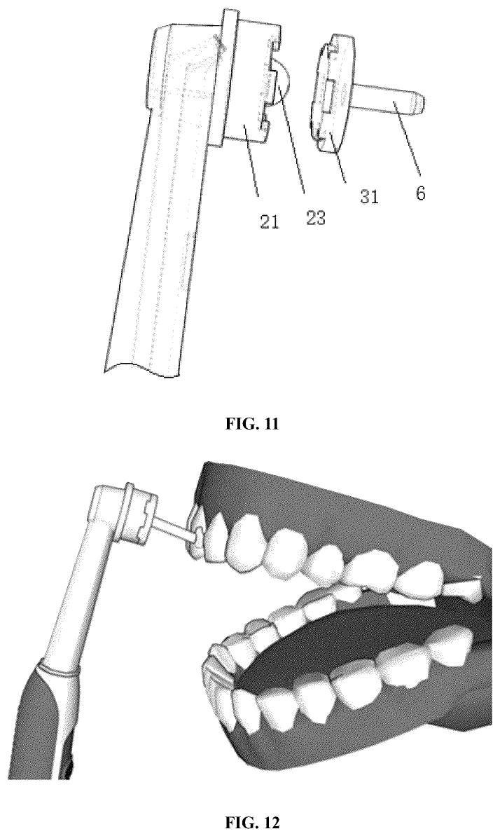

As shown in FIG. 7 to FIG. 12, a device for dental caries plaque cleaning and periodontitis sterilization is also provided, which includes a charger 1, a housing 2, a battery 4, a control chip 22, a light source 23, and a periodontal light guide needle 6. The battery 4, the control chip 22 and the light source 23 are encapsulated in the housing 2 to form a device main body. The housing 2 is provided with a working switch 24, a treatment time selection button 25, and a light beam selection button 26. The working switch 24, the treatment time selection button 25 and the light beam selection button 26 are all electrically connected to the control chip 22.

The periodontal light guide needle 6 is mounted at the head of the housing 2 using a mortise-tenon structure. A head structure of the housing 2 includes a connector 21, the light source 23 is arranged at the center of the connector 21, and the light source 23 is electrically connected to the control chip 22 and the battery 4. The periodontal light guide needle 6 further includes a mounting joint 31, which is in mortise-tenon connection and in interference fit with the connector 21 through the mounting joint 31.

The connector 21 is provided with four sections of clamping grooves, and the mounting joint 31 is correspondingly provided with four sections of grooves, and the clamping grooves are clamped into the grooves, and the connector 21 is connected to or disconnected from the mounting joint 31 in an interference manner.

The periodontal light guide needle 6 is mainly used for dental caries plaque cleaning and periodontitis treatment. The periodontal light guide needle 6 is made of polyvinyl chloride, is free of generating harmful substances during the treatment, low in cost, and capable of being designed and produced in large quantities, so as to achieve the purpose of disposable use. A tail end of the needle is designed with an interference type clamping groove, so as to be connected to or disconnected from the head of the device, and a front end of the needle is designed to be cylindrical. A long periodontal light guide needle and a short periodontal light guide needle are provided, which are suitable for internal and external periodontal diseases, respectively, thus facilitating treatment.

The rated optical power of the light source 23 is 25 mW. The light source 23 is a red-blue variable light source, where a wavelength of the red light ranges from 630 nm to 650 nm, and a wavelength of the blue light ranges from 430 nm to 450 nm. In general, a 3535RGB lamp bead and the control chip are employed.

The front end of the periodontal light guide needle 6 is cylindrical, the periodontal light guide needle 6 has a length of 5 cm or 2 cm, and is used for converging the light source. The optical power at a cylindrical tip is from 12.8 mW or 15.4 mW. Therefore, it is convenient for the photosensitizer entering the root canal of tooth to play a sterilization role.

The housing 2 has a height of about 17 cm, and a weight of about 150 g, and is made of polyvinyl chloride (PVC).

The periodontal light guide needle 6 is made of bisphenol A carbonate. A long periodontal light guide needle and a short periodontal light guide needle are provided, which are used for the treatment of internal and external periodontal diseases, respectively.

The light source 23 is a variable LED lamp bead, which is hemispherical, and the light beam is emitted around. After connecting the periodontal light guide needle 6, part of the light beam is converged on the needle tip, and part of the light beam is lost in the propagation process.

For dental caries plaque and periodontitis, the treatment power may be between 11 mW and 16 mW. Due to the existence of optical power loss, after being replaced repeatedly, a 3535RGB lamp bead with rated optical power of 25 mW and the control chip are finally used.

A relationship between a device supply voltage U (V) and the output power P (W) of the periodontal light guide needle 6 is as follows:

$$log ( P ) = 0.53 + 0.78 log ( U ) .$$

A relationship between the output power P (W) of the periodontal light guide needle 6 and the efficiency E (%) of killing Actinobacillus actinomycetemcomitans is as follows:

$$E = \frac{133}{1 + [ {( P / 10 )}^{- 1.5} ]} + 20.$$

### Embodiment 5

The device of Embodiment 4 is used for superficial caries treatment. Normal saline is first injected into caries plaque for rinsing, and the device is removed from a charger, and a disposable periodontal light guide needle is taken out. The short periodontal light guide needle is used for external periodontal, and the long periodontal light guide needle is used for internal periodontal. The periodontal light guide needle is mounted at the head of the device, and a working switch 3 on the housing is pressed for 3 s to start the device. A light beam selection button on the housing is pressed until an upper indicator lamp shows red, indicating that the selected beam wavelength is red light of 630 nm. A treatment time selection button on the housing is pressed until a lower indicator lamp shows white, indicating that the working time of the light source is 10 s.

The periodontal light guide needle is close to the caries plaque, the working switch is pressed to start the light source for photodynamic sterilization. The sterilization process is completed after 10 s, and then the light source is automatically turned off.

The light beam selection button on the housing is pressed until the upper indicator lamp shows blue, indicating that the selected beam is blue light with a wavelength of 450 nm, and then the caries plaque is irradiated again for 10 s. The above operation is repeated for three times, then the device is taken out from the oral cavity after the light source is automatically turned off, the working switch on the housing is pressed for 3 s to turn off the device. Finally, the inherent shape and function of the tooth are restored by filling a material or using other specific ways.

### Embodiment 6

The device of Embodiment 4 is used for treating deep caries. The device is removed from a charger, and then a disposable periodontal light guide needle is taken out and mounted on the head of the device, where the short periodontal light guide needle is used for external periodontal, and the long periodontal light guide needle is used for internal periodontal. The working switch on the housing is pressed for 3 s to start the device. The light beam selection button on the housing is pressed until the upper indicator lamp shows red, indicating that the selected beam is red light with a wavelength of 630 nm. The treatment time selection button on the housing is pressed until the lower indicator lamp shows yellow, indicating that the light source works for 30 s. 0.1 mg/ml toluidine blue O photosensitizer is injected into a deep caries cavity with a syringe, the periodontal light guide needle deeps into the deep caries cavity, the working switch is pressed to start the light source for photodynamic sterilization, after 30 seconds, the sterilization process is completed, the light source is automatically turned off, and the device is taken out from the oral cavity. The working switch on the housing is pressed for 3 s to turn off the device. Finally, the inherent shape and function of the tooth are restored by filling a material or using other specific ways.

### Embodiment 7

The device of Embodiment 4 and photosensitizer toluidine blue O are used in cooperation to carry out photodynamic sterilization of Actinobacillus actinomycetemcomitans, and a red light beam with a wavelength of 630 nm is selected to irradiate for 30 s. Experimental results show that the sterilization effect of a tip of periodontal light guide needle is obvious, and the sterilization efficiency can reach 99%.

### Embodiment 8

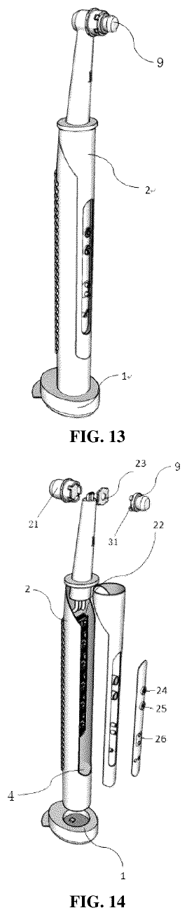

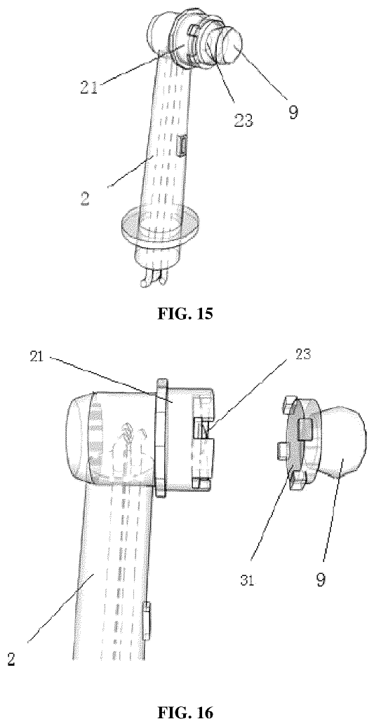

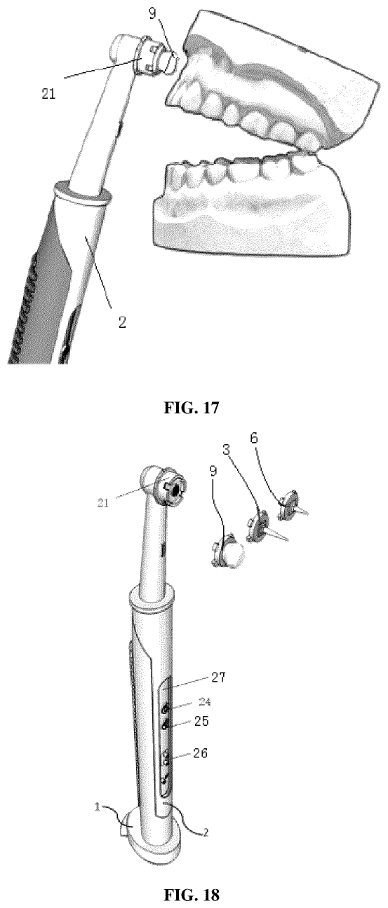

As shown in FIG. 13 to FIG. 17, the present disclosure provides a device for peri-implantitis and apical periodontitis elimination, including a housing 2, a battery 4, a control chip 22, a light source 23, and a pure flat light guide needle 9.

The battery 4 and the control chip 22 are encapsulated in the housing 2 to form a device main body. The battery 4 is encapsulated at the bottom inside the housing 2. The control chip 22 is encapsulated at a handle inside the housing 2. A working panel of the handle of the housing 2 is further provided with a working switch 24, a treatment time selection button 25, and a light beam selection button 26. The working switch 24, the treatment time selection button 25 and the light beam selection button 26 are all electrically connected to the control chip 22.

A head structure of the housing 2 includes a connector 211. The light source 23 is arranged on the connector 22, and the light source 23 is electrically connected to the control chip 22 and the battery 4.

The pure flat light guide needle 9 is mounted on the connector 21 using a mortise-tenon structure. A front end of the pure flat light guide needle 9 is in the shape of a frustum of a cone. The pure flat light guide needle has a length of 2 cm, and is used for irradiating the light source.

After several illumination experiments, it is found that part of the light emitted by the lamp beads (light source) will be scattered into the environment in actual use, resulting in loss. Therefore, the actual optical power of the pure flat light guide needle is from 12 mW to 16 mW, leading to a situation that the optical power of the lamp bead is greater than that of the light guide needle.

A timer is arranged in the control chip 22, and a switch is linked to a power supply wire of the lamp bead (light source). When the lamp bead is powered on, the timer starts to work, and when the timing reaches 2 min, the switch can be turned off immediately.

The device further includes a charger 1. The charger 1 is arranged at the bottom of the housing 2 and embedded with the housing 2. The charger 1 provides an electrode to be electrically connected to the battery 4.

The bottom of the pure flat light guide needle 9 is further provided with a mounting joint 31, which is in mortise-tenon connection and in interference fit with the connector 21 through the mounting joint 31.

The connector 21 is provided with four sections of clamping grooves, and the mounting joint 31 is correspondingly provided with four sections of grooves, and the clamping grooves are clamped into the grooves. The connector 21 is connected to or disconnected from the mounting joint 31 in an interference manner.

The housing 2 is made of polyvinyl chloride (PVC), is free of generating harmful substances during the treatment, low in cost, and capable of being designed and produced in large quantities, so as to achieve the purpose of disposable use.

A tail end is designed with an interference type clamping groove to be connected to and disconnected from the head of the device. A front end is designed as a frustum of cone. The light guide needle is used for irradiating the lesions of periodontitis and periapical periodontitis.

The rated optical power of the light source 23 is 25 mW. The light source 23 is a red-blue variable light source, where a wavelength of the red light ranges from 630 nm to 650 nm, and a wavelength of the blue light ranges from 430 nm to 450 nm. The light source 23 is an LED light source. In general, a 3535RGB lamp bead and the control chip are employed.

The front end of the pure flat light guide needle 9 is in the shape of a frustum of a cone, the pure flat light guide needle has a length of 2 cm, and is used for irradiating the light source. The optical power of the pure flat light guide needle 9 is from 12 mW to 16 mW, thus facilitating to directly irradiate a large-area lesion to control the photosensitizer to play a sterilization role.

The housing 2 has a height of about 17 cm and a weight of about 150 g, and is made of polyvinyl chloride (PVC).

The pure flat light guide needle 9 is made of bisphenol A carbonate, i.e., PC, which is used for irradiating the lesions of the periodontitis and periapical periodontitis.

The light source 23 is a variable LED lamp bed, which is hemispherical, and the light beam is emitted around. After connecting the pure flat light guide needle 9, part of the light beam converges on the needle tip, and part of the light beam is lost in the propagation process.

For dental caries plaque and periodontitis, the treatment power may be between 11 mW and 16 mW. Due to the existence of optical power loss, after being replaced repeatedly, the 3535RGB lamp bead with rated optical power of 25 mW and the control chip are finally used.

A relationship between a supply voltage U (V) of the battery 4 and the output power P (W) of the periodontal light guide needle 9 is as follows:

$$log ( p ) = 0.45 + 0.83 log ( U ) .$$

A relationship between the output power P (W) of the pure flat light guide needle 9 and the efficiency E (%) of killing Porphyromonas gingivalis is as follows:

$$E = \frac{133}{1 + {( \frac{1}{p} )}^{3 / 2}} + 20.$$

### Embodiment 9

The device of Embodiment 8 is used to treat periapical periodontitis in clinic. First, the lesion is cut open, drained, and rinsed, and blood and pus are sucked, and then then 0.1 mg/ml toluidine blue O photosensitizer is injected into a lesion site.

The device is removed from the charger, a disposable pure flat light guide needle is taken out and mounted on the head of the device, and the working switch 3 on the housing is pressed for 3 s to start the device. The light beam selection button on the housing is pressed until the upper indicator lamp shows red, indicating that the selected beam is red light with a wavelength of 630 nm. The treatment time selection button on the housing is pressed until the lower indicator lamp shows white, indicating that the working time of the light source is 10 s.

The pure flat light guide needle is close to the lesion, the working switch is pressed to start the light source for photodynamic sterilization. The sterilization process is completed after 30 s, and the light source is automatically turned off.

The device is taken out from the oral cavity. The working switch 3 on the housing is pressed for 3 s to turn off the device. Finally, the incision is rinsed, disinfected and closed.

### Embodiment 10

The device of Embodiment 8 is used to treat periodontitis in clinic. First, a lesion site is rinsed, and blood and pus are sucked, and then 0.1 mg/ml toluidine blue O photosensitizer is injected into the lesion site.

The device is removed from the charger, then the disposable pure flat light guide needle is taken out and mounted on the head of the device, the working switch 3 on the housing is pressed for 3 s to start the device. The light beam selection button on the housing is pressed until the upper indicator lamp shows red, indicating that the selected beam is red light with a wavelength of 630 nm. The treatment time selection button on the housing is pressed until the lower indicator lamp shows white, indicating that the working time of the light source is 10 s.

The pure flat light guide needle is close to the lesion, the working switch is pressed to start the light source for photodynamic sterilization. The sterilization process is completed after 30 s, and the light source is automatically turned off.

The device is taken out from the oral cavity. The working switch 3 on the housing is pressed for 3 s to turn off the device.

### Embodiment 11

The device of Embodiment 8 and photosensitizer toluidine blue O are used in cooperation to carry out photodynamic sterilization of Porphyromonas gingivalis, and a red light beam with a wavelength of 630 nm is selected to irradiate for 30 s. Experimental results show that the sterilization effect at a tail end part of the pure flat light guide needle is obvious, and the sterilization efficiency can reach 99%.

### Embodiment 12

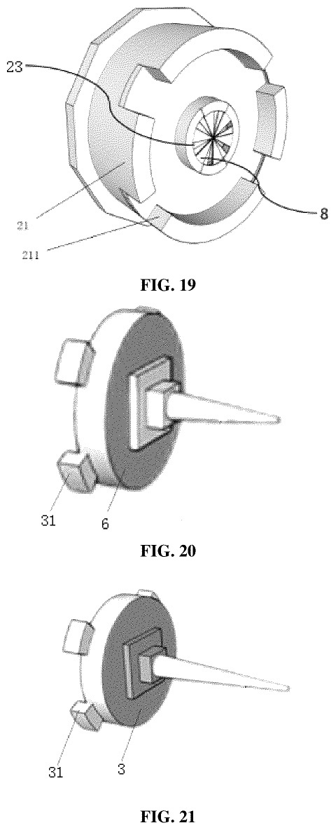

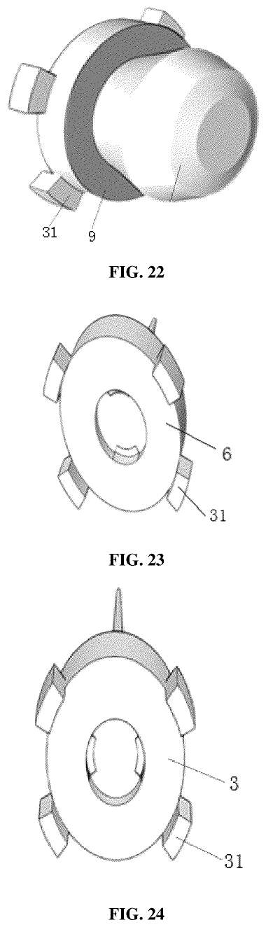

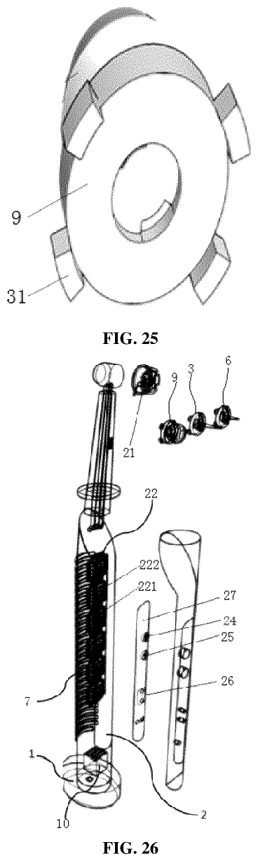

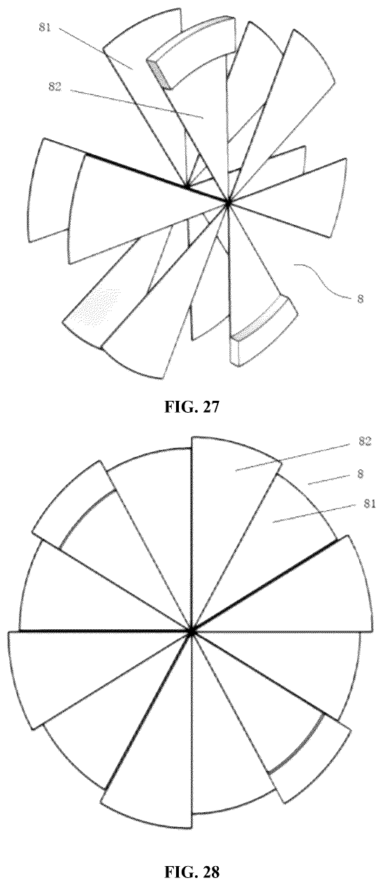

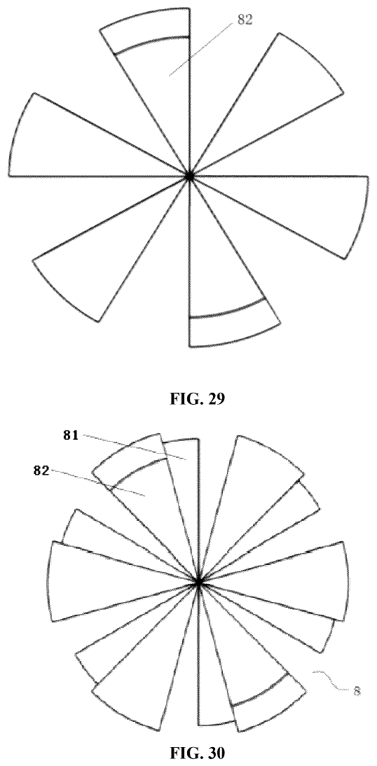

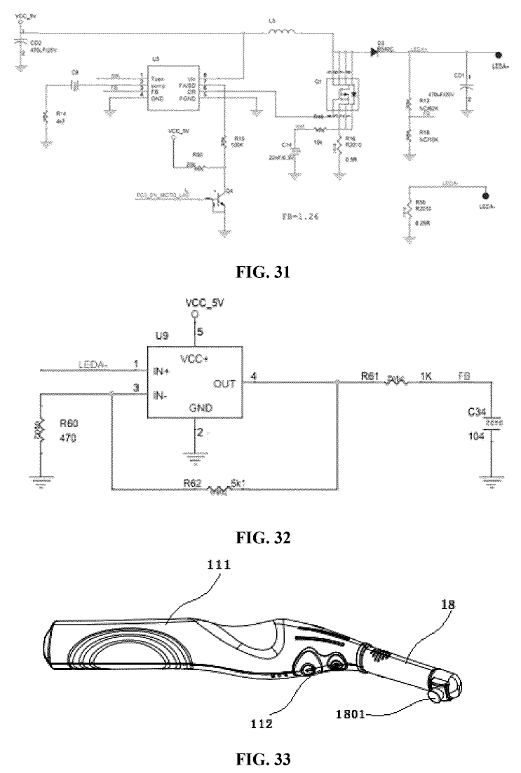

As shown in FIG. 18 to FIG. 32, an optical power constant-current source control device for oral treatment is also provided, including a charger 1, a housing 2, a constant-current source circuit 10, a control chip 22, a light source 23, a light guide needle 6, a control circuit 7, and a power attenuation sheet 8. The light source 23 is an LED light source.

The constant-current source circuit 10, the control chip 22 and the control circuit 7 are encapsulated in the housing 2. The housing 2 is made of a plastic material. A hand-held section of the housing 2 is provided with a control region 27 including a working switch 24, a treatment time selection button 25, and a light beam selection button 26. The control region 27 is electrically connected to the control chip 22.

The top of the housing 2 is provided with a concave connector 21, the light source 23 is arranged at the center of the connector 21, and is electrically connected to the working switch 24, the light beam selection button 26 and the control chip 22.

A front end of the light source 23 is fixedly provided with a power attenuation sheet 8. The power attenuation sheet 8 is of a double-layer structure, and includes an inner fixed sheet 81 and an outer rotary sheet 82 in coaxial arrangement. Each of the inner fixed sheet 81 and the outer rotary sheet 82 is composed of six 30° fan-shaped blades rotating in array at an interval of 30°. The inner fixed sheet 81 is fixed to the front end of the light source 23, the outer rotary sheet 82 rotates around the center of the inner fixed sheet 81 by 0-30°, and forms three different types of shading regions for the light source 23 with the inner fixed sheet 81, which are complete shading, partial shading and minimum shading, and thus three different output power states are formed to achieve regulation of the optical power.

Three models of light guide needles with different specifications and applications are provided, i.e., a short periodontal light guide needle 6, a long root canal light guide needle 3, and a pure flat light guide needle 9.

The optical power of the periodontal light guide needle 6 is from 60 mW to 85 mW, and the power attenuation sheet 8 is in partial shading. The optical power of the root canal light guide needle 3 is about 40 mW to 50 mW, and the power attenuation sheet 8 is in complete shading. The optical power of the pure flat light guide needle 9 is from 160 mW to 220 mW, and the power attenuation sheet 8 is in minimum shielding.

The light guide needle is fixedly mounted on a connector 21.

The periodontal light guide needle 6 is used for superficial treatment such as periodontitis and dental plaque irradiation. The root canal light guide needle 3 is used for irradiation operations such as dental pulp and root canals that require precise treatment. The pure flat light guide needle 9 is used for operations requiring large-scale irradiation for sterilization and anti-inflammatory such as periapical periodontitis and peri-implantitis.

An edge of the concave connector 21 is a circular arc, the circular arc is designed with at least two clamping grooves 211, and the light guide head is in mortise-tenon connection and in interference fit with the clamping grooves 211 through a corresponding convex mounting joint 31.

The rated output optical power of the constant-current source circuit 10 is from 500 mW to 700 mW. The light source 23 is a red-blue variable light source, where a wavelength of the red light ranges from 630 nm to 650 nm, and a wavelength of the blue light ranges from 430 nm to 450 nm.

The control chip 22 further includes a current control chip 222. The current control chip 222 is used for detecting a current in the circuit. When the current is excessive, information about the current is fed back to the control chip 22 to turn off the switch, so as to switch off the power supply circuit. Therefore, excessive life loss of the battery is avoided, and the service life of equipment is prolonged.

The control chip 22 further includes a temperature control chip 221 and an indicator lamp, the temperature control chip 221 is linked with the indicator lamp through a switch, and a timer starts to work when the indicator lamp is powered on. When the temperature control chip 221 monitors that a temperature is greater than 80° C., information about the temperature is fed back to the control chip 22 to turn off the switch, so as to switch off the power supply circuit. After the temperature control chip 221 monitors that the temperature drops to 40° C., information about the temperature is fed back to the control chip 22 to turn on the switch to restore the power supply of the power supply circuit, which makes the indicator lamp flash to prompt continued use.

The light guide needle may be made of bisphenol A carbonate. The short periodontal light guide needle 6 is used for superficial treatment such as periodontitis and dental plaque irradiation. The long root canal light guide needle 3 is used for irradiation operations such as dental pulp and root canals that require precise treatment. The pure flat light guide needle 9 is used for operations requiring large-scale irradiation for sterilization and anti-inflammatory such as periapical periodontitis and peri-implantitis.

### Embodiment 13

When the device of Embodiment 12 is used for clinical treatment of dental plaque, the tooth is first rinsed and dried with biological cotton, and then photosensitizer toluidine blue O (TBO) is applied to the tooth surface.

The device is removed from the charger, then the short periodontal light guide needle is mounted, the working switch on the housing is pressed for 3 s to start the device. The light beam selection button on the housing is pressed until the upper indicator lamp shows red, indicating that the selected beam is red light with the wavelength of 630 nm. The treatment time selection button on the housing is pressed, the root canal light guide needle is close to the lesion, the working switch is pressed to start the light source for photodynamic sterilization, and after 30 s, the sterilization process is completed, the light source is automatically turned off, and the device is taken out from the oral cavity. The working switch on the housing is pressed for 3 s to turn off the device.

### Embodiment 14

When the device of Embodiment 12 is used for clinical treatment of the root canal, the tooth is first rinsed and dried with biological cotton, and then the photosensitizer toluidine blue O (TBO) is injected into the root canal.

The device is removed from the charger, then the long root canal light guide needle is mounted, the working switch on the housing is pressed for 3 s to start the device. The light beam selection button on the housing is pressed until the upper indicator lamp shows blue, indicating that the selected beam wavelength is blue light of 430 nm. The treatment time selection button on the housing is pressed, the root canal light guide needle is close to the lesion, the working switch is pressed to start the light source for photodynamic sterilization, and after 60 s, the sterilization process is completed, the light source is automatically turned off, and the device is taken out from the oral cavity. The working switch on the housing is pressed for 3 s to turn off the device.

### Embodiment 15

The device of Embodiment 12 is used for clinical treatment of periapical periodontitis, firstly, a lesion site is drained and rinsed to suck up blood and pus, and then toluidine blue O (TBO) is injected into a gingival lesion site.

The device is removed from the charger, then the disposable pure flat light guide needle is taken out and mounted at the head of the device. The working switch 3 on the housing is pressed for 3 s to start the device. The light beam selection button on the housing is pressed until the upper indicator lamp shows blue, indicating that the selected beam wavelength is blue light of 430 nm. The pure flat light guide needle is close to the lesion, the working switch is pressed to start the light source for photodynamic sterilization, and after 90 s, the sterilization process is completed, the light source is automatically turned off, and the device is taken out from the oral cavity. The working switch 3 on the housing is pressed for 3 s to turn off the device, and finally, the incision is rinsed, disinfected and closed.

### Embodiment 16

The device of Embodiment 12 is used for clinical treatment of periodontitis. First, a lesion site is rinsed to suck up the blood and pus, and then toluidine blue O (TBO) photosensitizer is injected into the lesion site. The device is removed from the charger, then the disposable pure flat light guide needle is taken out and mounted at the head of the device. The working switch 3 on the housing is pressed for 3 s to start the device. The light beam selection button on the housing is pressed until the upper indicator lamp shows blue, indicating that the selected beam wavelength is blue light of 430 nm. The treatment time selection button on the housing is pressed, the pure flat light guide needle is close to the lesion, the working switch button is pressed to start the light source for photodynamic sterilization, and after 90 s, the sterilization process is completed, the light source is automatically turned off, and the device is taken out from the oral cavity. The working switch 3 on the housing is pressed for 3 s to turn off the device.

### Embodiment 17

The device of Embodiment 12 is used for normal root canal filling and cleaning, if the patient does not have special circumstances such as the lesion, the blue light is used to directly irradiate the lesion, without using toluidine blue O for cooperation, where it is indicated that the system is ready when observing that the indicator lamp turns blue and always lights up. The light beam selection button on the housing is pressed until the upper indicator lamp shows blue, indicating that the selected beam wavelength is blue light of 430 nm. The treatment time selection button on the housing is pressed, the pure flat light guide needle is close to the lesion, the working switch button is pressed to start the light source for photodynamic sterilization, and after 90 s, the sterilization process is completed, the light source is automatically turned off, and the device is taken out from the oral cavity. The working switch on the housing is pressed for 1 s to turn off the device.

### Embodiment 18

The device of Embodiment 12 and photosensitizer toluidine blue O are used in cooperation to carry out the photodynamic sterilization experiment on Porphyromonas gingivalis and Enterococcus faecalis. A red light beam with a wavelength of 630 nm and a blue light beam with a wavelength of 430 nm are used to irradiate for 30 seconds. The experimental results show that the sterilization effect at a tail end of the device is obvious, and the sterilization efficiency can reach 99%.

### Embodiment 19

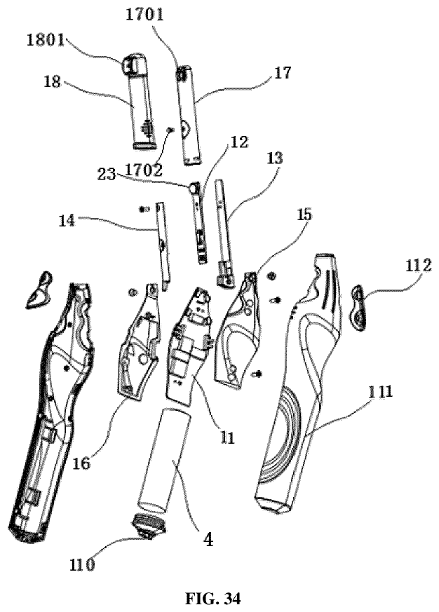

As shown in FIG. 33 and FIG. 34, an oral hygiene device provided by the present disclosure is a device for oral photodynamic therapy, including a main board 11, a metal substrate 12, a light source 23, a heat dissipation plate 13, a heat dissipation cover 14, a first cooling fin 15, a second cooling fin 16, a light source sleeve 17, a working sleeve 18, a battery 4, a charging interface 110, a lower housing 111, and a control button 112.

The main board 11 is provided with a control chip, and a control circuit. The main board 11 is used to control the light source 23. The light source 23 may be an LED lamp source.

The metal substrate 12 is electrically connected to the main board 11. The metal substrate 12 is provided with a light source mounting surface. The light source 23 is located on the light source mounting surface. The light source 23 is arranged at one end, away from the main board 11, of the metal substrate 12.

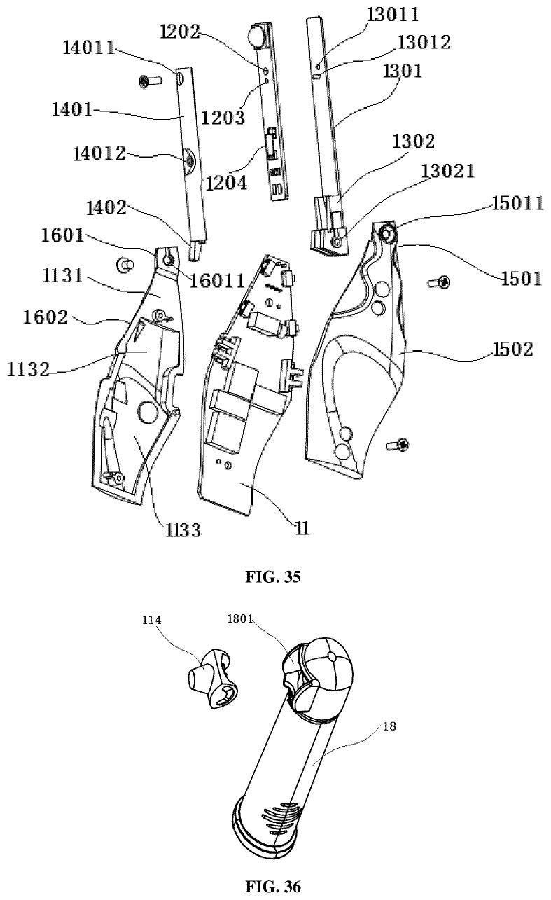

As shown in FIG. 35, the metal substrate 12 is provided with a first metal substrate connecting hole 1202, and a second metal substrate connecting hole 1203 near the light source 23. The metal substrate 12 may be made of aluminum. The light source 23 can emit blue light and red light.

In some other embodiments, the heat dissipation plate 13 is provided to dissipate and conduct the heat generated by the light source 23 to the first cooling fin 15 and the second cooling fin 16.

As shown in FIG. 35, the heat dissipation plate 13 is provided with a heat dissipation plate body 1301, and a heat dissipation plate connecting part 1302. The heat dissipation plate body 1301 is connected to the heat dissipation plate connecting part 1302. The heat dissipation plate body 1301 and the heat dissipation plate connecting part 1302 may be of an integrated structure.

As shown in FIG. 35, the heat dissipation plate body 1301 is attached to a surface, opposite to the light source mounting surface, of the metal substrate 12. The light source mounting surface coincides with a projection of the heat dissipation plate body 1301 on a plane where the light source mounting surface is located. The heat dissipation plate body 1301 is provided with a first heat dissipation plate connecting hole 13011, and a heat dissipation plate connecting pillar 13012. The heat dissipation plate connecting pillar 13012 is in fit with the second metal substrate connecting hole 1203, thereby fixing opposite positions of the heat dissipation plate 13 and the metal substrate 12.

As shown in FIG. 35, in a direction perpendicular to the light source mounting surface, a thickness of the heat dissipation plate connecting part 1302 is greater than that of the heat dissipation plate body 1301, thus making the heat dissipation plate connecting part 1302 form a bulge on one side where the metal substrate 12 is located. The metal substrate 12 may be arranged on the bulge formed by the heat dissipation plate connecting part 1302. The heat dissipation plate connecting part 1302 is provided with a second heat dissipation plate connecting hole 13021.

The heat dissipation plate 13 is made of metal.

In some other embodiments, the heat dissipation cover 14 is provided to dissipate and conduct the heat generated by the light source 23 to the first cooling fin 15 and the second cooling fin 16.

As shown in FIG. 35, the heat dissipation cover 14 is provided with a heat dissipation cover body 1401, and a heat dissipation cover connecting part 1402. The heat dissipation cover body 1401 is connected to the heat dissipation cover connecting part 1402. The heat dissipation cover body 1401 and the heat dissipation cover connecting part 1402 may be of an integrated structure.

The heat dissipation cover body 1401 may cover on the light source mounting surface, without shading the light source 23. The light source mounting surface coincides with a projection of the heat dissipation cover body 1401 on the plane where the light source mounting surface is located. The heat dissipation cover body 1401 is provided with a heat dissipation cover connecting hole 14011.

The heat dissipation cover connecting part 1402 is connected to the heat dissipation plate connecting part 1302. The metal substrate 12, the heat dissipation plate 13 and the heat dissipation cover 14 are connected together through the first metal substrate connecting hole 1202, the first heat dissipation plate connecting hole 13011, and the heat dissipation cover connecting hole 14011. Specifically, a first fixing screw passes through the first metal substrate connecting hole 1202, the first heat dissipation plate connecting hole 13011 and the heat dissipation cover connecting hole 14011, thereby connecting the metal substrate 12, the heat dissipation plate 13 and the heat dissipation cover 14 together. Therefore, the metal substrate 12 is fixed between the heat dissipation cover 14 and the heat dissipation plate 13.

As shown in FIG. 35, the first cooling fin 15 is located on one side of the main board 11. The surface area of the first cooling fin 15 is greater than that of the heat dissipation plate 13, and is greater than that of the heat dissipation cover 14. In some embodiments, the first cooling fin 15 is used to dissipate the heat conducted by the heat dissipation plate 13 and the heat dissipation cover 14. The first cooling fin 15 is provided with a first cooling fin connecting part 1501, and a first cooling fin main board accommodating part 1502. The first cooling fin connecting part 1501 is connected to the first cooling fin main board accommodating part 1502. The first cooling fin connecting part 1501 and the first cooling fin main board accommodating part 1502 may be of an integrated structure.

The second cooling fin 16 is located on the other side of the main board 11. The surface area of the second cooling fin 16 is greater than that of the heat dissipation plate 13, and is greater than that of the heat dissipation cover 14. The first cooling fin 15 and the second cooling fin 16 are located on two opposite sides of the main board 11, respectively. In some embodiments, the second cooling fin 16 is used to dissipate the heat conducted by the heat dissipation plate 13 and the heat dissipation cover 14. The second cooling fin 16 is provided with a second cooling fin connecting part 1601, and a second cooling fin main board accommodating part 1602. The second cooling fin connecting part 1601 is connected to the second cooling fin main board accommodating part 1602. The second cooling fin connecting part 1601 and the second cooling fin main board accommodating part 1602 may be of an integrated structure.

The first cooling fin connecting part 1501 is provided with a first cooling fin connecting hole 15011. The second cooling fin connecting part 1601 is provided with a second cooling fin connecting hole 16011. The heat dissipation plate 13, the first cooling fin 15 and the second cooling fin 16 are connected together through the first cooling fin connecting hole 15011, the second cooling fin connecting hole 16011, and the second heat dissipation plate connecting hole 13021. Specifically, the first cooling fin 15 and the second cooling fin 16 are located on both sides of the heat dissipation plate 13, respectively. A second fixing screw passes through the first cooling fin connecting hole 15011, the second cooling fin connecting hole 16011 and the second heat dissipation connecting hole 13021 to connect the first cooling fin 15, the second cooling fin 16 and the heat dissipation plate 13.

The first cooling fin main board accommodating part 1502 and the second cooling fin main board accommodating part 1602 are oppositely arranged to form a main board accommodating cavity. The main board 11 is located in the main board accommodating cavity. The main board 11 is fixedly connected to the first cooling fin main board accommodating part 1502 and the second cooling fin main board accommodating part 1602.

The main board accommodating cavity is provided with a first cavity part 1131, a second cavity part 1132, and a third cavity part 1133 which are in communication in turn. The first cavity part 1131 is close to the first cooling fin connecting part 1501 and the second cooling fin connecting part 1601. In the first cavity part 1131, a distance between the first cooling fin 15 and the second cooling fin 16 is denoted by F. In the second cavity part 1132, a distance between the first cooling fin 15 and the second cooling fin 16 is denoted by S. In the third cavity part 1133, a distance between the first cooling fin 15 and the second cooling fin 16 is denoted by T, T is less than S and is greater than or equal to F.

The first cooling fin 15 and the second cooling fin 16 are both made of metal.

An elastic sheet 1204 is arranged near one end of the metal substrate 12 close to the main board 11. The elastic sheet 1204 is located on the light source mounting surface. The elastic sheet 1204 is used to control main board 11 to be connected to and disconnected from the light source 23.

As shown in FIG. 35, the heat dissipation cover body 1401 is provided with an elastic sheet opening 14012 at a position in fit with the elastic sheet 1204. At least a part of a projection of the elastic sheet opening 14012 on the plane where the light source mounting surface is located coincides with a projection of the elastic sheet 1204 on the plane where the light source mounting surface is located. In some embodiments, the projection of the elastic sheet opening 14012 on the plane where the light source mounting surface is located falls within the range of the projection of the elastic sheet 1204 on the plane where the light source mounting surface is located.

As shown in FIG. 34, the light source sleeve 17 is sleeved outside the metal substrate 12, the heat dissipation plate 13, and the heat dissipation cover 14. The light source sleeve 17 is provided with a light source hole 1701, and an elastic sheet button 1702.

The light generated by the light source 23 can pass through the light source hole 1701. The area of a projection of the light source hole 1701 on the plane where the light source mounting surface is located is greater than that of a projection of the light source 23 on the plane where the light source mounting surface is located. In some embodiments, the center of the projection of the light source hole 1701 on the plane where the light source mounting surface is located coincides with the center of the projection of the light source 23 on the plane where the light source mounting surface is located.

At least a part of elastic sheet button 1702 is arranged on an outer surface of the light source sleeve 17 and protrudes to an outside of the light source sleeve 17. The elastic sheet button 1702 can move to an elastic sheet 1204 direction under the action of an external force. A projection of the elastic sheet button 1702 on the plane where the light source mounting surface is located falls within the range of the projection of the elastic sheet opening 14012 on the plane where the light source mounting surface is located. The elastic sheet button 1702 touches the elastic sheet 1204 through the elastic sheet opening 14012, thereby compressing the elastic sheet 1204 and making the main board 11 communicate with the light source 23.

The working sleeve 18 is provided with a connector. The connector is connected to the light guide needle. The connector is provided with a connector light-transmitting hole 1801. The light generated by the light source 23 can pass through the connector light-transmitting hole 1801. The area of the projection of the connector light-transmitting hole 1801 on the plane where the light source mounting surface is located is greater than that of the projection of the light source 23 on the plane where the light source mounting surface is located. When the working sleeve 18 is sleeved on the periphery of the light source sleeve 17, the projection of the light source 23 on the plane where the light source mounting surface is located falls within the range of the projection of the connector light-transmitting hole 1801 on the plane where the light source mounting surface is located. Moreover, due to the extrusion effect of the working sleeve 18 on the elastic sheet button 1702, the elastic sheet button 1702 moves to the direction of the elastic sheet button 1204, thereby compressing the elastic sheet 1204 and making the light source 23 communicate with the main board 11.

As shown in FIG. 34, the battery 4 is electrically connected to the main board 11. The battery 4 stores electric energy and supplies the electric energy to the main board 11.

The charging interface 110 is electrically connected to the battery 4. The charging interface 110 supplies electric energy to the battery 4. The charging interface 110 is provided with a power supply connecting terminal, and the power supply connecting terminal is connected to an external power supply.

The battery 4, the main board 11, the first cooling fin 15, the second cooling fin 16 and the charging interface 110 are located in a cavity formed by the lower housing 111. The lower housing 111 is provided with a housing opening, through which the power supply connecting terminal is exposed outside. The working sleeve 18 is connected to the lower housing 111.

The control button 112 is arranged on the lower housing 111, and the control button 112 is connected to the control chip. The control button 112 controls the lighting and extinguishing of the light source 23, the lighting time of the light source 23 and the wavelength of the light emitted by the light source 23.

### Embodiment 20

Except for the following structure, the rest is the same as in Embodiment 19.

The oral hygiene device of this embodiment further includes a light guide needle 114. As shown in FIG. 36, the light guide needle 114 is connected to the connector. The light guide needle 114 focuses the light source emitted through the connector light-transmitting hole 1801 on a site to be treated.

### Embodiment 21

An oral hygiene system of this embodiment includes the device of Embodiment 20 and a compound shown in Formula (I):

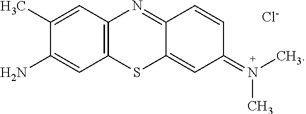

The above compound can excite singlet oxygen when cooperating with the red light or blue light emitted by the device of the present disclosure, so as to achieve good sterilization and anti-inflammation. In addition, compared with laser, the red light and blue light emitted by the device of the present disclosure have lower power and less damage to teeth and surrounding tissues.

The present disclosure is not limited to the above embodiments, and any variation, improvement and substitution that can be thought of by those skilled in the art fall within the scope of the present disclosure without departing from the essence of the present disclosure.

What is claimed is:

1. An oral hygiene device, comprising a housing, a battery, a control chip, a light source, and a light guide needle;
wherein the battery, the control chip and the light source are encapsulated in the housing to form a device main body;
the head of the housing is provided with a connector, the light source is arranged at the center of the connector, and the light source is electrically connected to the control chip and the battery;
the light guide needle is mounted at the head of the housing using a mortise-tenon structure, and the light guide needle is provided with a mounting joint, and is in mortise-tenon connection and in interference fit with the connector through the mounting joint.

2. The device according to claim 1, wherein
the light source is a red-blue variable light source, a wavelength of red light ranges from 630 nm to 650 nm, and a wavelength of blue light ranges from 430 nm to 450 nm, and the light source is an LED (light-emitting diode) working light source;
the light guide needle is made of bisphenol A polycarbonate, and is selected from one of the following light guide needles:
(1) a root canal light guide needle, wherein a front end of the root canal light guide needle is cone-shaped, and optical power at a conical tip is 4.7 mW;
(2) a periodontal light guide needle, wherein a front end of the periodontal light guide needle is cylindrical and is used for converging the light source, and optical power at a cylindrical tip is 12.8 mW or 15.4 mW; and
(3) a pure flat light guide needle, wherein a front end of the pure flat light guide needle is in the shape of a frustum of a cone.

3. The device according to claim 1, further comprising a power attenuation sheet, wherein the power attenuation sheet is arranged at a front end of the light source;
the power attenuation sheet is of a double-layer structure, comprises an inner fixed sheet and an outer rotary sheet in coaxial arrangement; each of the inner fixed sheet and the outer rotary sheet is composed of six 30° fan-shaped blades rotating in an array at intervals of 30° from each other; the inner fixed sheet is fixed to the front end of the light source, the outer rotary sheet rotates around the center of the inner fixed sheet by 0-30°, and forms three different types of shading regions for the light source with the inner fixed sheet, which are complete shading, partial shading and minimum shading, and thus three different output power states are formed to achieve regulation of the optical power.

4. The device according to claim 3, wherein three models of light guide needles with different specifications and applications are provided, i.e., a short periodontal light guide needle, a long root canal light guide needle, and a pure flat light guide needle;
optical power of the periodontal light guide needle is from 60 mW to 85 mW, and the power attenuation sheet is in partial shading; optical power of the root canal light guide needle is about 40 mW to 50 mW, and the power attenuation sheet is in complete shading; and optical power of the pure flat light guide needle is from 160 mW to 220 mW, and the power attenuation sheet is in minimum shielding.

5. The device according to claim 4, wherein the device further comprises a constant current circuit, which is encapsulated in the housing; and rated output optical power of the constant current circuit is from 500 mW to 700 mW.

6. The device according to claim 4, wherein the device further comprises a current control chip, and/or a temperature control chip;
the current control chip is used for detecting a current in a power supply circuit, and when the current is excessive, information about excessive current is fed back to the control chip to turn off the switch, so as to switch off the power supply circuit;
the temperature control chip is linked with an indicator lamp through a switch; when the temperature control chip monitors that a temperature of the device is greater than 80° C., information about the temperature is fed back to the control chip to turn off the switch, so as to switch off the power supply circuit; after the temperature control chip monitors that the temperature of the device drops to 40° C., information about the temperature is fed back to the control chip to turn on the switch to restore the power supply of the power supply circuit, which makes the indicator lamp flash to prompt continued use.

7. The device according to claim 1, wherein the device further comprises a main board, a metal substrate, a heat dissipation plate, a heat dissipation cover, a first cooling fin, and a second cooling fin;
the main board is provided with a control chip, and a control circuit;
the metal substrate is provided with a light source mounting surface, the light source is arranged on the light source mounting surface, and the metal substrate is electrically connected to the main board;
the heat dissipation plate is in contact with a surface, opposite to the light source mounting surface, of the metal substrate;
the heat dissipation cover is in contact with the light source mounting surface; and
the first cooling fin and the second cooling fin are arranged on two opposite sides of the main board, respectively.

8. The device according to claim 7, further comprising a working sleeve, wherein the housing comprises a lower housing, and a light source sleeve;
the metal substrate is provided with an elastic sheet, and the elastic sheet is provided to control the main board to connect to and disconnect from the light source;
the heat dissipation cover is provided with an elastic sheet opening, at least a part of a projection of the elastic sheet opening on a plane where the light source mounting surface coincides with a projection of the elastic sheet on the plane where the light source mounting surface;
the light source sleeve is sleeved outside the metal substrate, the heat dissipation plate and the heat dissipation cover; the light source sleeve is provided with a light source hole, and an elastic sheet button, the light source hole is provided for the transmission of light generated by the light source, and the elastic sheet button is arranged to touch the elastic sheet under the action of an external force;
the working sleeve is provided with a connector, and the connector is provided with a connector light-transmitting hole; the connector light-transmitting hole is provided for the transmission of light generated by the light source; and when the working sleeve is sleeved on the periphery of the light source sleeve, the working sleeve touches the elastic sheet button; and
the main board, the first cooling fin, the second cooling fin and the battery are arranged in the lower housing.

9. An oral hygiene system, comprising:
an oral hygiene device, wherein the oral hygiene device comprises a housing, a battery, a control chip, a light source, and a light guide needle; wherein the battery, the control chip and the light source are encapsulated in the housing to form a device main body, a head of the housing includes a connector, the light source is positioned in the center of the connector, and the light source is electrically connected to the control chip and the battery; the light guide needle is mounted at the head of the housing using a mortise-tenon structure, and the light guide needle is provided with a mounting joint, and is in mortise-tenon connection and in interference fit with the connector through the mounting joint; and
a compound shown in following Formula: 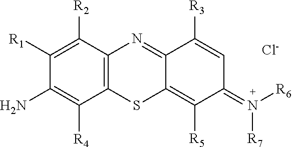
wherein R₁, R₂, R₃, R₄, R₅, R₆ and R₇ are independently selected from H or C1-C6 alkyl, respectively.

10. The oral hygiene system according to claim 9, wherein the light source is a red-blue variable light source, a wavelength of red light ranges from 630 nm to 650 nm, and a wavelength of blue light ranges from 430 nm to 450 nm, and the light source is an LED (light-emitting diode) working light source;
the light guide needle is made of bisphenol A polycarbonate, and is selected from one of the following light guide needles:
(1) a root canal light guide needle, wherein a front end of the root canal light guide needle is cone-shaped, and optical power at a conical tip is 4.7 mW;
(2) a periodontal light guide needle, wherein a front end of the periodontal light guide needle is cylindrical and is used for converging the light source, and optical power at a cylindrical tip is 12.8 mW or 15.4 mW; and
(3) a pure flat light guide needle, wherein a front end of the pure flat light guide needle is in the shape of a frustum of a cone.

11. The oral hygiene system according to claim 9, further comprising a power attenuation sheet, wherein the power attenuation sheet is positioned at a front end of the light source;
the power attenuation sheet is of a double-layer structure, comprises an inner fixed sheet and an outer rotary sheet in coaxial arrangement; each of the inner fixed sheet and the outer rotary sheet is composed of six 30° fan-shaped blades rotating in an array at intervals of 30° from each other; the inner fixed sheet is fixed to the front end of the light source, the outer rotary sheet rotates around the center of the inner fixed sheet by 0-30°, and forms three different types of shading regions for the light source with the inner fixed sheet, which are complete shading, partial shading and minimum shading, and thus three different output power states are formed to achieve regulation of the optical power.

12. The oral hygiene system according to claim 11, wherein three models of light guide needles with different specifications and applications are provided, i.e., a short periodontal light guide needle, a long root canal light guide needle, and a pure flat light guide needle;
optical power of the periodontal light guide needle is from 60 mW to 85 mW, and the power attenuation sheet is in partial shading; optical power of the root canal light guide needle is about 40 mW to 50 mW, and the power attenuation sheet is in complete shading; and optical power of the pure flat light guide needle is from 160 mW to 220 mW, and the power attenuation sheet is in minimum shielding.

13. The oral hygiene system according to claim 12, wherein the device further comprises a constant current circuit, which is encapsulated in the housing; and rated output optical power of the constant current circuit is from 500 mW to 700 mW.

14. The oral hygiene system according to claim 12, wherein the device further comprises a current control chip, and/or a temperature control chip;
the current control chip is used for detecting a current in a power supply circuit, and when the current is excessive, information about excessive current is fed back to the control chip to turn off the switch, so as to switch off the power supply circuit;
the temperature control chip is linked with an indicator lamp through a switch; when the temperature control chip monitors that a temperature of the device is greater than 80° C., information about the temperature is fed back to the control chip to turn off the switch, so as to switch off the power supply circuit; after the temperature control chip monitors that the temperature of the device drops to 40° C., information about the temperature is fed back to the control chip to turn on the switch to restore the power supply of the power supply circuit, which makes the indicator lamp flash to prompt continued use.

15. The oral hygiene system according to claim 9, wherein the device further comprises a main board, a metal substrate, a heat dissipation plate, a heat dissipation cover, a first cooling fin, and a second cooling fin;
the main board is provided with a control chip, and a control circuit;
the metal substrate is provided with a light source mounting surface, the light source is arranged on the light source mounting surface, and the metal substrate is electrically connected to the main board;
the heat dissipation plate is in contact with a surface, opposite to the light source mounting surface, of the metal substrate;
the heat dissipation cover is in contact with the light source mounting surface; and
the first cooling fin and the second cooling fin are arranged on two opposite sides of the main board, respectively.

16. The oral hygiene system according to claim 15, further comprising a working sleeve, wherein the housing comprises a lower housing, and a light source sleeve;
the metal substrate is provided with an elastic sheet, and the elastic sheet is provided to control the main board to connect to and disconnect from the light source;
the heat dissipation cover is provided with an elastic sheet opening, at least a part of a projection of the elastic sheet opening on a plane where the light source mounting surface coincides with a projection of the elastic sheet on the plane where the light source mounting surface;
the light source sleeve is sleeved outside the metal substrate, the heat dissipation plate and the heat dissipation cover; the light source sleeve is provided with a light source hole, and an elastic sheet button, the light source hole is provided for the transmission of light generated by the light source, and the elastic sheet button is arranged to touch the elastic sheet under the action of an external force;
the working sleeve is provided with a connector, and the connector is provided with a connector light-transmitting hole; the connector light-transmitting hole is provided for the transmission of light generated by the light source; and when the working sleeve is sleeved on the periphery of the light source sleeve, the working sleeve touches the elastic sheet button; and
the main board, the first cooling fin, the second cooling fin and the battery are arranged in the lower housing.

17. A method of using an oral hygiene device, comprising:
irradiating teeth or tissue in an oral cavity using red light and/or blue light.

18. The method according to claim 17, wherein the oral hygiene device comprises: a housing, a battery, a control chip, a light source, and a light guide needle;
wherein the battery, the control chip and the light source are encapsulated in the housing to form a device main body;
the head of the housing is provided with a connector, the light source is arranged at the center of the connector, and the light source is electrically connected to the control chip and the battery;
the light guide needle is mounted at the head of the housing using a mortise-tenon structure, and the light guide needle is provided with a mounting joint, and is in mortise-tenon connection and in interference fit with the connector through the mounting joint.

19. The method according to claim 18, wherein the light source is a red-blue variable light source, a wavelength of red light ranges from 630 nm to 650 nm, and a wavelength of blue light ranges from 430 nm to 450 nm, and the light source is an LED (light-emitting diode) working light source;
the light guide needle is made of bisphenol A polycarbonate, and is selected from one of the following light guide needles:
(1) a root canal light guide needle, wherein a front end of the root canal light guide needle is cone-shaped, and optical power at a conical tip is 4.7 mW;
(2) a periodontal light guide needle, wherein a front end of the periodontal light guide needle is cylindrical and is used for converging the light source, and optical power at a cylindrical tip is 12.8 mW or 15.4 mW; and
(3) a pure flat light guide needle, wherein a front end of the pure flat light guide needle is in the shape of a frustum of a cone.

20. The method according to claim 18, wherein the oral hygiene device further comprises: a power attenuation sheet, wherein the power attenuation sheet is positioned at a front end of the light source, the power attenuation sheet is of a double-layer structure, comprises an inner fixed sheet and an outer rotary sheet in coaxial arrangement; each of the inner fixed sheet and the outer rotary sheet is composed of six 30° fan-shaped blades rotating in an array at intervals of 30° from each other; the inner fixed sheet is fixed to the front end of the light source, the outer rotary sheet rotates around the center of the inner fixed sheet by 0-30°, and forms three different types of shading regions for the light source with the inner fixed sheet, which are complete shading, partial shading and minimum shading, and thus three different output power states are formed to achieve regulation of the optical power.
